(19) United States
(12) Patent Application Publication
(10) Pub. No.: US 2013/0261460 A1
(43) Pub. Date: Oct. 3, 2013

(54) ULTRASONIC PROCESSING APPARATUS AND PROBE SUPPORTING APPARATUS

(71) Applicant: SONY CORPORATION, TOKYO (JP)

(72) Inventor: Tatsumi Sakaguchi, Kanagawa (JP)

(73) Assignee: SONY CORPORATION, TOKYO (JP)

(21) Appl. No.: 13/779,902

(22) Filed: Feb. 28, 2013

(30) Foreign Application Priority Data
Mar. 30, 2012 (JP) 2012-082536

(51) Int. Cl. A61B 8/00; A61B 8/13

(52) U.S. Cl. CPC A61B 8/4455; A61B 8/13; A61B 8/4245
USPC 600/443; 600/459

(57) ABSTRACT

An ultrasonic processing apparatus includes a probe, a supporting unit that is provided at an angle perpendicular to a beam direction of the probe, a rotation mechanism that is provided between the probe and the supporting unit, and a guide that supports the rotating operation of the probe by the rotation mechanism.

### BACKGROUND

The present disclosure relates to an ultrasonic processing apparatus and a probe supporting apparatus, and particularly to an ultrasonic processing apparatus and a probe supporting apparatus which enable a three-dimensional structure of a target portion to be easily and precisely achieved.

In an ultrasonic apparatus which performs capturing an ultrasonic image, detecting the motion of a probe plays an important role in the processes of computer aided diagnosis, measurements of tissue form or characterization, panoramic image generation, 3D reconstruction, or the like.

For the detection of probe motion, for example, in Japanese Unexamined Patent Application Publication No. 2005-185333, a method has been proposed in which two scanning surfaces are formed by a two-dimensional probe, and motion detection and three-dimensional movement reconstruction of the probe are performed. Moreover, in Japanese Unexamined Patent Application Publication No. 2010-227603, a method has been proposed in which an ultrasonic probe is formed by one-dimensional array oscillators that are perpendicular to each other and tracing of ultrasonic probe motion is performed.

Since it is possible to perform a non-invasive examination with ease, the ultrasonic apparatuses described above have been commonly used instead of X-rays or MRI, in a case where there is a necessity for the observation of joints of extremities such as rheumatoid arthritis examination.

In this case, the same ultrasonic apparatus, probe, and imaging process as an ultrasonic apparatus, a probe, and an imaging process, which are originally used for observing a heart or an abdominal part, are used.

### SUMMARY

However, in rheumatoid arthritis examination or the like, it is necessary to monitor a joint over a long period of time. An ultrasonic diagnosis apparatus has characteristics such as a low reproducibility (a case where it is difficult to observe the same affected part from the same place at a different timing). Therefore, the ultrasonic diagnosis apparatus is not suitable for uses such as a follow-up observation over a long period of time.

As described above, there is a demand for a method to acquire a three-dimensional structure at one time, when performing the observation of joints.

It is desirable to easily and precisely achieve a three-dimensional structure of a target portion.

According to a first embodiment of the present disclosure, there is provided an ultrasonic processing apparatus including a probe, a supporting unit that is provided at an angle perpendicular to a beam direction of the probe, a rotation mechanism that is provided between the probe and the supporting unit, and a guide that supports the rotating operation of the probe by the rotation mechanism.

The guide may be provided on the probe at a right angle to a sensor surface of the probe.

The guide may be provided on the probe so as to adjust a distance between the center of the sensor surface of the probe and the guide to be the radius of a diagnosis target object.

The probe may rotate around the rotation mechanism as an axis so as to rotate the guide in the opposite direction to the sensor surface of the probe.

The guide may be provided on the same plane with a sensor surface of the probe.

The guide may be provided at an angle perpendicular to the beam direction of the probe.

The guide may be provided on a rotational direction side of the probe.

The guide may be provided on an opposite direction side to the rotational direction of the probe.

The length of the guide in the rotational direction of the probe may be longer than the length of the guide in the opposite direction side.

The length of the guide in the rotational direction of the probe may be the same as the length of the guide in the opposite direction side.

The supporting unit may be provided on the probe so as to be at 90 degrees to the beam direction of the probe.

The supporting unit may include an auxiliary operation unit having a rotation mechanism.

The rotation mechanism of the auxiliary operation unit may be prohibited from rotating about the rotational axis of the rotation mechanism.

The auxiliary operation unit may be detachably provided.

The probe may include an angle sensor detecting an angle of the probe.

The probe may include a movement amount sensor measuring a movement amount of a sensor surface on a body surface.

The ultrasonic processing apparatus may further include an information acquisition unit that acquires information representing a position of the probe by which ultrasonic waves generation and reflective waves reception are performed, and a cross-sectional image generation unit that generates a tomographic image representing at least a part of the cross sections of a subject to be imaged, by arranging and synthesizing a plurality of ultrasonic images which are based on reflective waves received by the probe at a plurality of positions around the subject to be imaged, based on an angle of the probe when ultrasonic waves generation and reflective waves reception are performed.

The ultrasonic processing apparatus may further include a probe state detection unit that detects a state of the probe based on information acquired by the information acquisition unit. The information acquisition unit may acquire data representing the position of the probe from a plurality of types of sensors, and the probe state detection unit may select data to be used for detecting the state of the probe, among data acquired by the plurality of sensors.

The ultrasonic processing apparatus may further include an image generation unit that generates a plurality of simplified display images corresponding to a plurality of ultrasonic images which are arranged at a position interlocking with rotating operation of the probe in virtual space and are inputted from the probe, and a display control unit that controls displaying of the plurality of simplified display images which are generated by the image generation unit and are arranged at a position interlocking with the rotating operation of the probe.

The ultrasonic processing apparatus may further include a signal processing unit that processes a signal received from a oscillator configuring the probe or a signal to be transmitted to the oscillator, and a control unit that controls a signal processing parameter so as to increase the parameter of the signal processing unit when a rotational angle of the probe is small.

The ultrasonic processing apparatus may further include a signal processing unit that processes a signal received from a oscillator configuring the probe or a signal to be transmitted to the oscillator, and a control unit that controls the signal processing unit so as to transmit a signal to the oscillator when a rotational angle of the probe is coincident with a predetermined imaging angle.

According to a second embodiment of the present disclosure, there is provided a probe supporting apparatus including a supporting unit that is provided at an angle perpendicular to a beam direction of a probe, a rotation mechanism that is provided between the probe and the supporting unit, and a guide that supports the rotating operation of the probe by the rotation mechanism.

According to the first embodiment of the present disclosure, there is provided a probe, a supporting unit that is provided at an angle perpendicular to a beam direction of the probe, a rotation mechanism that is provided between the probe and the supporting unit, and a guide that supports the rotating operation of the probe by the rotation mechanism.

According to the second embodiment of the present disclosure, there is provided a supporting unit that is provided at an angle perpendicular to a beam direction of a probe, a rotation mechanism that is provided between the probe and the supporting unit, and a guide that supports the rotating operation of the probe by the rotation mechanism.

According to the present disclosure, it is possible to easily and precisely achieve a three-dimensional structure of a target portion.

### BRIEF DESCRIPTION OF THE DRAWINGS

FIGS. 1A to 1C are views showing a configuration example of the appearance of an ultrasonic probe according to the present disclosure;

FIG. 2 is a view illustrating an operation of the ultrasonic probe;

FIG. 3 is a view illustrating an operation of the ultrasonic probe;

FIG. 4 is a view illustrating an operation of the ultrasonic probe;

FIG. 5 is a view illustrating an operation of the ultrasonic probe;

FIGS. 6A to 6C are views showing another configuration example of the appearance of the ultrasonic probe according to the present disclosure;

FIGS. 7A to 7C are views showing still another configuration example of the appearance of the ultrasonic probe according to the present disclosure;

FIGS. 8A to 8C are views showing further still another configuration example of the appearance of the ultrasonic probe according to the present disclosure;

FIGS. 9A and 9B are views showing further still another configuration example of the appearance of the ultrasonic probe according to the present disclosure;

FIG. 10 is a view illustrating a rotational direction of a ball joint;

FIG. 11 is a table showing the usability of jigs in the probe;

FIG. 12 is a block diagram showing a configuration example of an image processing system according to the present disclosure;

FIG. 13 is a flowchart illustrating imaging processes of the image processing system;

FIG. 14 is a flowchart illustrating an example of simplified display image group generation processes of the image processing system;

FIG. 15 is a flowchart illustrating another example of simplified display image group generation processes of the image processing system;

FIGS. 16A and 16B are views showing an example of a simplified display image group and virtual space arrangement thereof;

FIG. 17 is a view showing an example of the simplified display image group;

FIG. 18 is a view showing an example of the simplified display image group;

FIG. 19 is a view showing an example of the simplified display image group;

FIG. 20 is a view showing an example of virtual space arrangement;

FIG. 21 is a view showing another configuration example of the ultrasonic probe according to the present disclosure;

FIG. 22 is a view illustrating an image surface of an array oscillator;

FIG. 23 is a view illustrating an acoustic lens in the ultrasonic probe;

FIG. 24 is a view illustrating the effect of the acoustic lens in the x axis direction;

FIG. 25 is a view illustrating the effect of the acoustic lens in the z axis direction;

FIG. 26 is a view illustrating the calculation of a movement amount of the probe in the image processing system;

FIG. 27 is a view illustrating the application of a two-dimensional array probe of the present disclosure;

FIG. 28 is a block diagram showing another configuration example of the image processing system according to the present disclosure;

FIG. 29 is a block diagram showing a configuration example of a probe unit in a case where an ultrasonic wave receiving side process is performed;

FIG. 30 is a block diagram showing a configuration example of the probe unit in a case where an ultrasonic wave transmitting side process is performed;

FIG. 31 is a flowchart illustrating an example of ultrasonic wave reception processes of the probe unit;

FIG. 32 is a flowchart illustrating an example of a reception display process of a reception display device;

FIG. 33 is a flowchart illustrating an example of ultrasonic wave transmission processes of the probe unit;

FIG. 34 is a flowchart illustrating an example of processes before imaging of the image processing system;

FIG. 35 is a flowchart illustrating an example of imaging processes of the image processing system; and

FIG. 36 is a block diagram showing a configuration example of a computer.

### DETAILED DESCRIPTION OF EMBODIMENTS

Hereinafter, embodiments for carrying out the present disclosure (hereinafter, referred to as embodiments) will be described. In addition, the description will be made as follows.

1. First Embodiment (Ultrasonic Probe)

2. Second Embodiment (Image Processing System)

3. Third Embodiment (User Interface)

4. Fourth Embodiment (Other Configurations of Ultrasonic Probe)

5. Fifth Embodiment (Other Configurations of Image Processing System)

6. Sixth Embodiment (Computer)

### First Embodiment

### Configuration Example of Appearance of Ultrasonic Probe

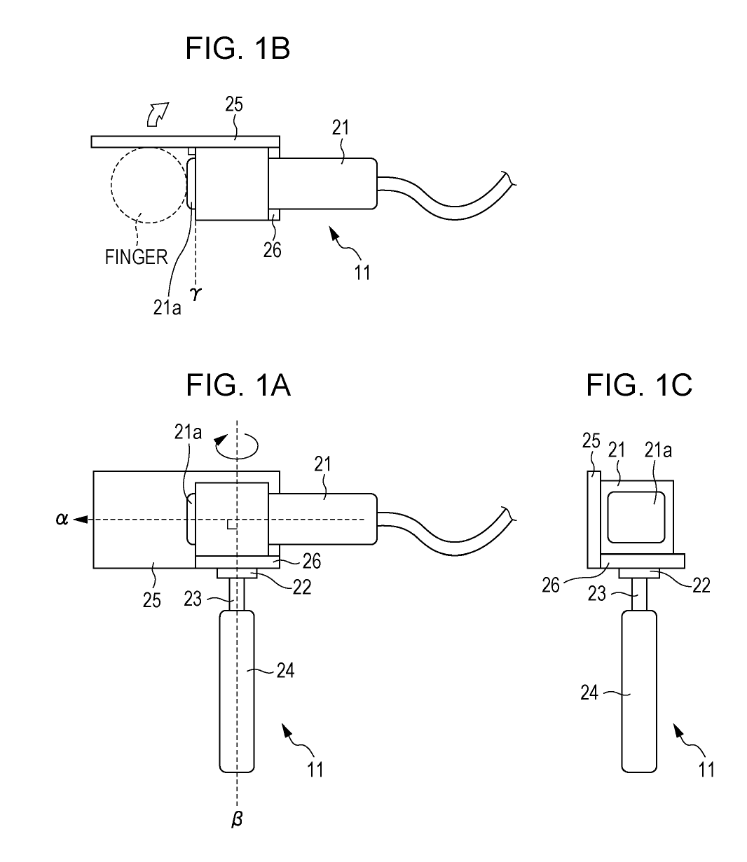

FIGS. 1A to 1C are views showing a configuration example of the appearance of an ultrasonic probe according to the present disclosure. FIG. 1A is a side view of the ultrasonic probe, FIG. 1B is a plan view of the ultrasonic probe shown in FIG. 1A when seen from the top, and FIG. 1C is a front view of the ultrasonic probe shown in FIG. 1A when seen from the left.

In the examples of FIGS. 1A to 1C, an ultrasonic probe 11 is configured to include a probe (main body) 21, a seat 22, a rotational axis 23, a handle 24, a guide 25, and a joint portion 26. In addition, the ultrasonic probe 11 in the examples of FIGS. 1A to 1C has a structure which is suitable for turning around a finger or the like as a diagnosis target, which is relatively thin.

The probe 21 is a sector probe, for example. However, the probe 21 may be a probe having other structures. The probe 21 has a contact portion 21a, which is formed by a flexible member and comes into contact with a diagnosis target object, on a sensor surface of the probe. The probe 21 is configured to arrange transducers in a vertical direction of the contact portion 21a in FIG. 1C, for example.

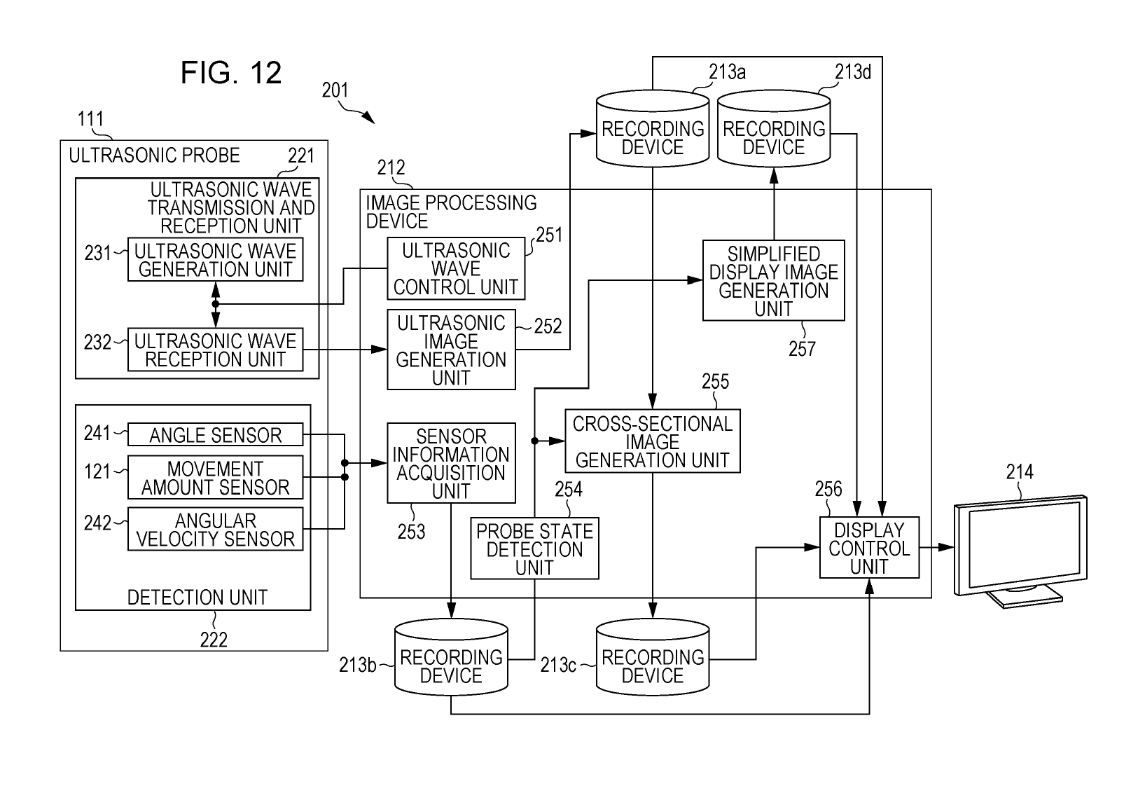

The seat 22 having a built-in angle sensor (an angle sensor 241 of FIG. 12 described later), the rotational axis 23 which is a rotation mechanism attached to the angle sensor, and the handle 24 are provided under the probe 21. The rotational axis 23 and the probe 21 are fixed by, for example, the joint portion 26, but the fixing method is not limited thereto.

A geomagnetic sensor or an accelerometer may be provided instead of an angle sensor. However, by attaching the angle sensor, rotational angle information of the probe 21 can be more precisely acquired compared to a geomagnetic sensor or an accelerometer.

The rotational axis 23 is provided between the probe 21 and the handle 24 such that the ultrasonic probe 11 rotates about the rotational axis 23 (an axis β perpendicular to a beam direction) as a center in a horizontal direction of the contact portion 21a in FIG. 1C, for example, when the ultrasonic probe 11 rotates about a finger. The handle is a supporting unit which a person to be imaged uses. The rotational axis 23 and the handle 24 are provided at an angle perpendicular to a beam direction indicated by the end of a center axis X of the sensor surface of the probe 21. In this way, it is possible to stably rotate the probe 21 around a diagnosis target object without using a human hand.

In the present specification, the definition of the angle perpendicular to a beam direction is not only limited to a strict 90 degree angle but may also include an angle having the operation function as described above. However, practically, it is preferable that a 90 degree angle be used.

The guide 25 is provided on a side surface of the probe 21 (the left side of the contact portion 21a in FIG. 1C) and the guide 25 supports the rotating operation such that an ultrasonic beam of the probe 21 is typically transmitted from a vertical direction with respect to a diagnosis target object when rotating the ultrasonic probe 11. The guide 25 is formed by a panel or the like and is provided on the probe 21 and the joint portion 26 such that the guide 25 is disposed at a right angle with respect to the same plane γ as the sensor surface of the probe 21. Moreover, in the present specification, the definition of the right angle is not limited to a 90 degree angle, but any angle may be used as long as it has a function to steadily come into contact therewith without displacing a center axis and it does not deteriorate a maneuvering feeling.

For example, a diagnosis target object such as a finger represented by dotted lines is allowed to come into contact with the contact portion 21a on the sensor surface and the guide 25 and the contact portion 21a and the guide 25 are rotated in the direction represented by a white arrow, that is, in the opposite direction to the sensor surface. In this way, the contact portion 21a on the sensor surface and the guide 25 fix the diagnosis target object. Therefore, the contact portion 21a and the guide 25 are stably rotated around the cylindrical diagnosis target object represented by dotted lines such that an ultrasonic beam almost vertically reaches the diagnosis target object at all times.

It is possible to rotate the guide 25 in the opposite direction to the direction represented by a white arrow. However, since there is a possibility that the diagnosis target object floats with respect to the contact portion 21a on the sensor surface and the guide 25, it is preferable to rotate the guide 25 in the direction represented by a white arrow.

In examples of FIGS. 1A to 1C, the sensor surface (the same plane γ as the sensor surface) is shown so as not to come into contact with the diagnosis target object. However, practically, since there is an elastic force between the contact portion 21a and the diagnosis target object and, moreover, at the time of diagnosis, imaging is performed after applying gel thereto, the sensor surface and the diagnosis target object is in a close contact state. The same is applied to the drawings described later.

Operation of Ultrasonic Probe

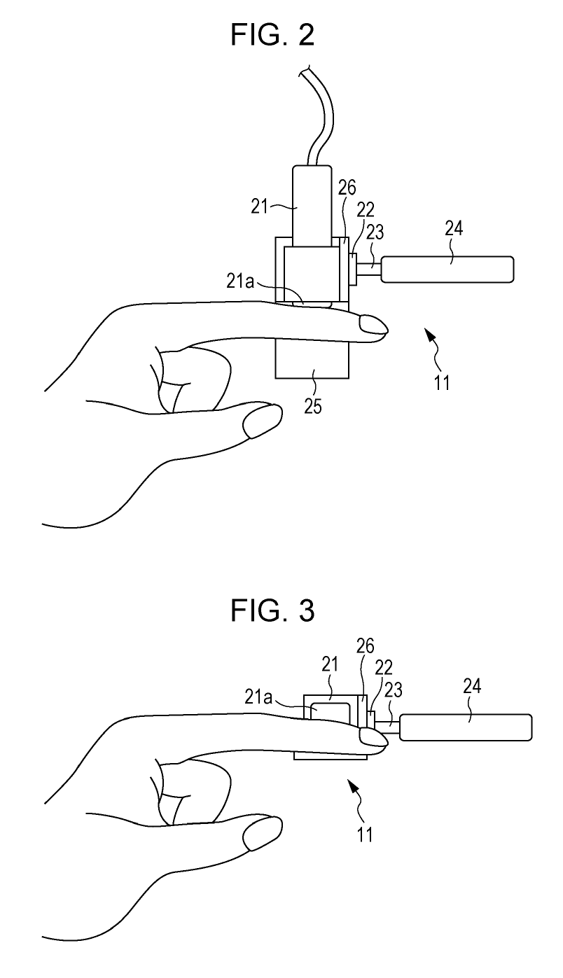

Next, with reference to FIGS. 2 to 5, the rotating operation of the ultrasonic probe 11 will be described. In examples of FIGS. 2 to 5, a hand of a person to be imaged who holds the handle 24 is omitted.

Hereinafter, the description will be made as regards to the rotating operation, but imaging is also performed while rotating. Regarding the rotating operation of the ultrasonic probe 11, the starting position thereof is set to a predetermined position of a diagnosis target object (for example, the back of a finger shown in FIG. 2), in advance.

As shown in FIG. 2, the person to be imaged allows the contact portion 21a on the sensor surface of the probe 21 and the guide 25 to come into contact with the back of his or her finger, which is a starting position, so as to start imaging. The ultrasonic probe 11 is rotated in the depth direction of the drawing so as to circle around the finger.

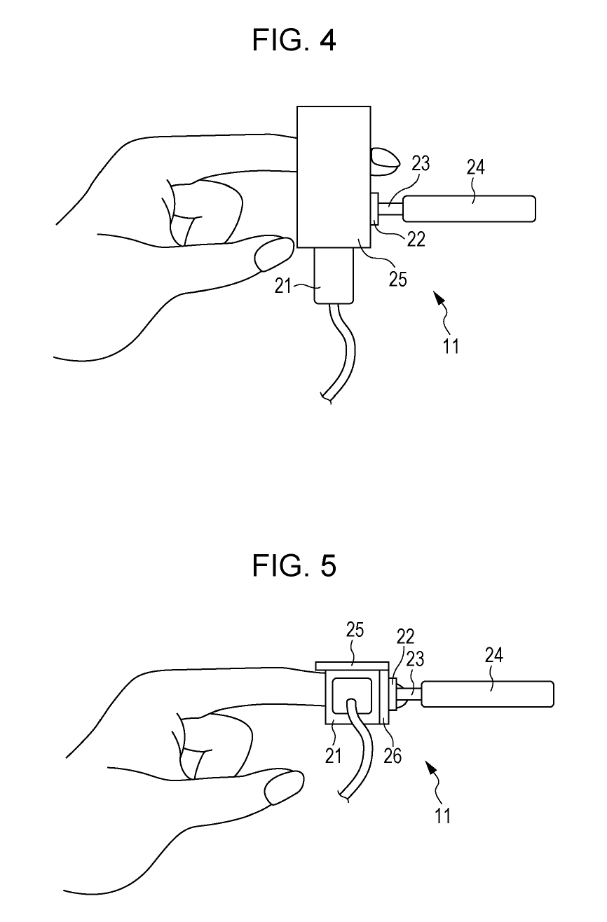

That is to say, while the probe 21 passes through, in this order, the back of the finger shown in FIG. 2, the left side of the finger shown in FIG. 3, the ball of the finger shown in FIG. 4, and the right side of the finger shown in FIG. 5, the probe 21 captures ultrasonic images thereof.

During the rotating operation around the finger, the guide 25 and the contact portion 21a on the sensor surface of the probe 21 are not separated from the surface of the finger and are not displaced. Therefore, it is possible to stably rotate the ultrasonic probe 11 while an ultrasonic beam is allowed to be transmitted in the vertical direction to the finger.

### Another Configuration Example of Appearance of Ultrasonic Probe

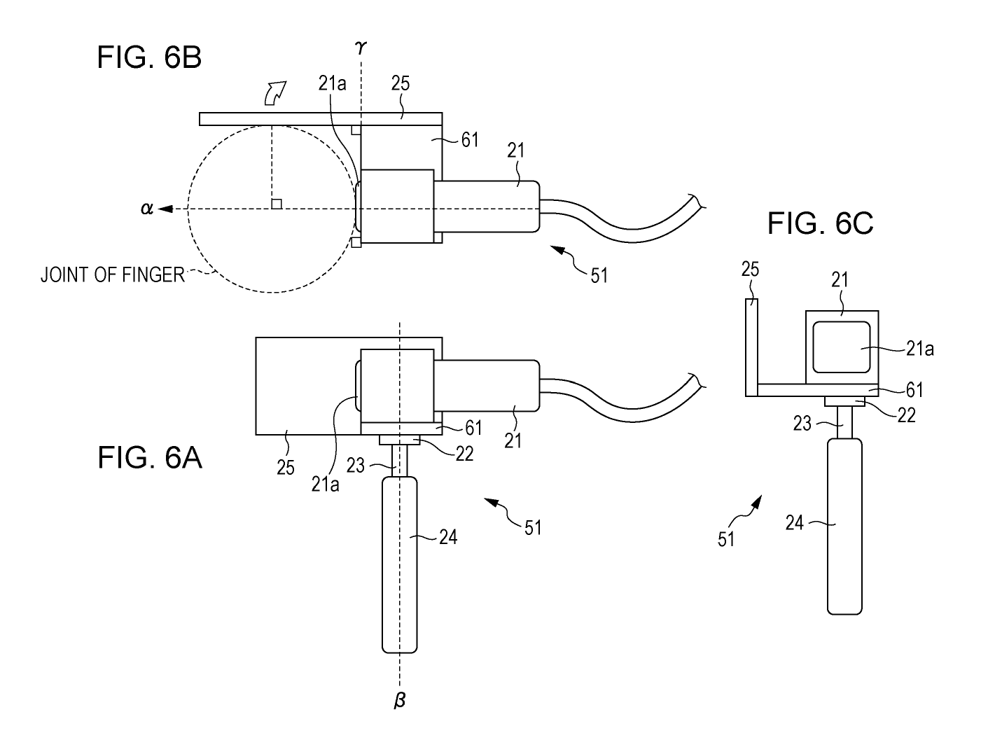

FIGS. 6A to 6C are views showing another configuration example of the appearance of an ultrasonic probe according to the present disclosure. FIG. 6A is a side view of the ultrasonic probe, FIG. 6B is a plan view of the ultrasonic probe shown in FIG. 6A when seen from the top, and FIG. 6C is a front view of the ultrasonic probe shown in FIG. 6A when seen from the left.

In examples of FIGS. 6A to 6C, an ultrasonic probe 51 has similarity with the ultrasonic probe 11 of FIGS. 1A to 1C in that the probe (main body) 21, the seat 22, the rotational axis 23, the handle 24, and the guide 25 are included. The ultrasonic probe 51 is different from the ultrasonic probe 11 of FIGS. 1A to 1C in that the joint portion 26 is switched with a joint portion 61.

That is, the joint portion 26 shown in FIGS. 1A to 1C is fixed on the probe 21, but the length of the joint portion 61 shown in FIGS. 6A to 6C is capable of being adjusted (variable) so as to provide a gap between the guide 25 and the probe 21.

The guide 25 can be provided on the probe 21 by the joint portion 61 such that a distance between the guide 25 and the center axis X of the sensor surface of the probe 21 is equivalent to the radius of the diagnosis target object. In other words, the guide 25 can be provided on the probe 21 such that the center axis α of the sensor surface of the probe 21 is equivalent to the center position of the diagnosis target object.

In this way, it is possible to cope with a thickish cylindrical diagnosis target object represented by dotted lines.

That is to say, in a similar way to the case of FIGS. 1A to 1C, a diagnosis target object such as a cubital joint represented by dotted lines is allowed to come into contact with the contact portion 21a on the sensor surface and the guide 25 and the contact portion 21a and the guide 25 are rotated in the direction represented by a white arrow, that is, in the opposite direction to the sensor surface. In this way, even in a thickish cylindrical diagnosis target object, the contact portion 21a on the sensor surface and the guide 25 fix the diagnosis target object. Therefore, the contact portion 21a and the guide 25 are stably rotated around the cylindrical diagnosis target object represented by dotted lines such that an ultrasonic beam almost vertically reaches the diagnosis target object at all times.

### Another Configuration Example of Appearance of Ultrasonic Probe

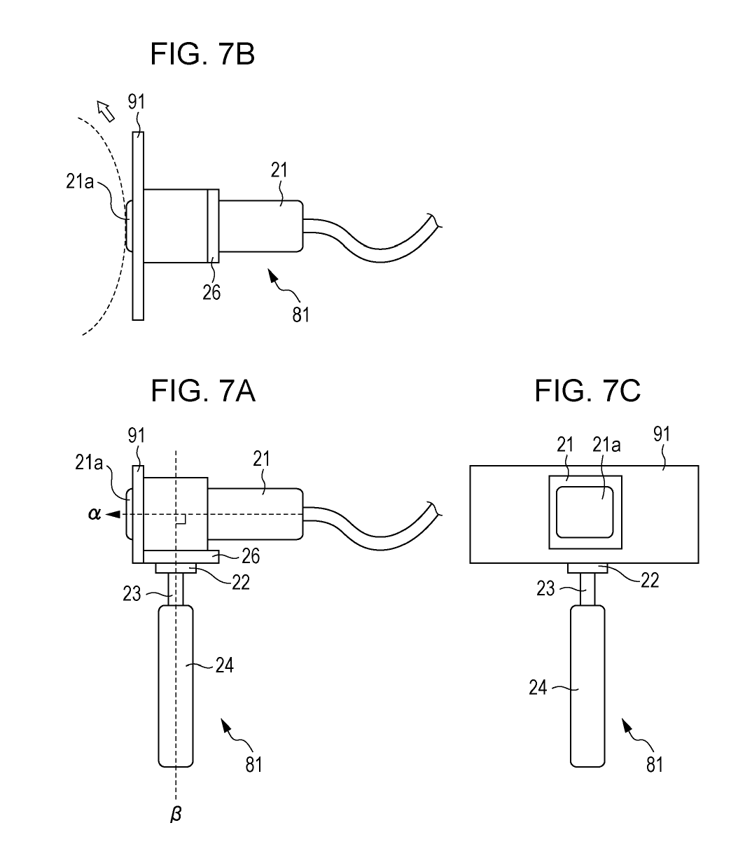

FIGS. 7A to 7C are views showing still another configuration example of the appearance of the ultrasonic probe according to the present disclosure. FIG. 7A is a side view of the ultrasonic probe, FIG. 7B is a plan view of the ultrasonic probe shown in FIG. 7A when seen from the top, and FIG. 7C is a front view of the ultrasonic probe shown in FIG. 7A when seen from the left.

In examples of FIGS. 7A to 7C, an ultrasonic probe 81 has similarity with the ultrasonic probe 11 of FIGS. 1A to 1C in that the probe (main body) 21, the seat 22, the rotational axis 23, the handle 24, and the joint portion 26 are included. The ultrasonic probe 81 is different from the ultrasonic probe 11 of FIGS. 1A to 1C in that the guide 25 is switched with a guide 91.

That is, the guide 91 is provided on a moving direction (rotational direction) side of the probe 21, which is the same surface as the sensor surface of the probe 21, at an angle perpendicular to a beam direction indicated by the end of the center axis α of the sensor surface of the probe 21. Moreover, one surface guide in a proceeding direction of the probe 21 may be used. However, as shown in FIG. 7B, the guide may not only be configured to extend in the proceeding direction (the upper direction in the drawing), but also be configured to extend in the opposite direction (the lower direction in the drawing) to the proceeding direction, thereby rotating more stably. In addition, in the present specification, the definition of the angle perpendicular to a beam direction is not only limited to a strict 90 degree angle but also may include an angle at which an ultrasonic beam almost vertically reaches the diagnosis target object. However, practically, it is preferable that a 90 degree angle be used.

In examples of FIGS. 7A to 7C, a case where the lengths of the guide 91 in the proceeding direction and in the opposite direction are equivalent to each other is shown. However, the length of the guide in the proceeding direction may be longer than that of the guide in the opposite direction.

Herein, the configurations of the ultrasonic probe 11 of FIGS. 1A to 1C and the ultrasonic probe 51 of FIGS. 6A to 6C, which are described above, are more effective in a case where the cross section of the diagnosis target object is close to a completely round circle, practically. However, in a case where the cross section of the diagnosis target object is an elliptical shape such as a wrist or an elbow, the configurations thereof are difficult to be applied thereto. Therefore, in a case where the cross section of the diagnosis target object is an elliptical shape, the configuration of the ultrasonic probe 81 of FIGS. 7A to 7C is suitable.

In a case of examples of FIGS. 7A to 7C, a diagnosis target object such as a cubital joint represented by dotted lines is allowed to come into contact with the guide 91 (including the contact portion 21a on the sensor surface) and the guide 91 is rotated in the direction represented by a white arrow. In this way, even in an elliptical-shaped cylindrical diagnosis target object, the guide 91 fixes the diagnosis target object. Therefore, the guide 91 is stably rotated around the elliptical-shaped cylindrical diagnosis target object represented by dotted lines such that an ultrasonic beam almost vertically reaches the diagnosis target object at all times.

In examples of FIGS. 7A to 7C, the guide 91 (sensor surface) is shown so as not to come into contact with the diagnosis target object. However, practically, since there is an elastic force between the contact portion 21a and the diagnosis target object and, moreover, at the time of diagnosis, imaging is performed after applying gel thereto, the guide 91 and the diagnosis target object are in a close contact state.

### Another Configuration Example of Appearance of Ultrasonic Probe

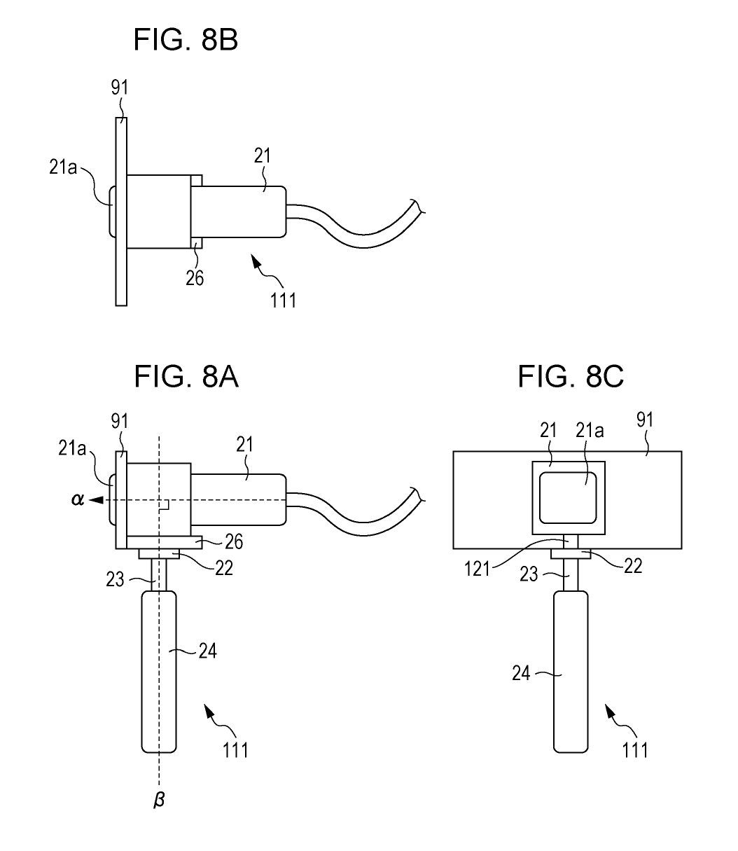

FIGS. 8A to 8C are views showing further still another configuration example of the appearance of the ultrasonic probe according to the present disclosure. FIG. 8A is a side view of the ultrasonic probe, FIG. 8B is a plan view of the ultrasonic probe shown in FIG. 8A when seen from the top, and FIG. 8C is a front view of the ultrasonic probe shown in FIG. 8A when seen from the left.

In examples of FIGS. 8A to 8C, an ultrasonic probe 111 has similarity with the ultrasonic probe 81 of FIGS. 7A to 7C in that the probe (main body) 21, the seat 22, the rotational axis 23, the handle 24, the joint portion 26, and the guide 91 are included. The ultrasonic probe 111 is different from the ultrasonic probe 81 of FIGS. 7A to 7C in that a movement amount sensor 121 is added to the probe 21.

That is, in examples of FIGS. 8A to 8C, the movement amount sensor 121 is provided below the probe 21 and on the guide 91. The setting position of the movement amount sensor 121 is not limited thereto. For example, the movement amount sensor 121 is configured by an optical movement amount sensor used for an optical mouse or the like, and detects the movement amount on the body surface such as sideslip other than the operation of the ultrasonic probe 111.

In the ultrasonic probe 111, in a case where the diagnosis target object is an elliptical-shaped cylindrical object or the like, operations such as sideslip are performed in addition to the rotating operation. In the ultrasonic probes 11, 51 and 81 described above, only the angle sensor is provided. However, in order to reconstruct a three-dimensional volume in this condition, the restriction on which the diagnosis target object is moved on the circumference at the uniform velocity is necessary.

In a case of the ultrasonic probe 111, the movement amount sensor 121 is provided, thereby removing the restriction.

In the examples of FIGS. 8A to 8C, the example in which the movement amount sensor 121 is provided on the ultrasonic probe 81 of FIGS. 7A to 7C has been described. However, needless to say, the movement amount sensor 121 can be provided on ultrasonic probes having other configurations, such as the ultrasonic probe 11 of FIGS. 1A to 1C or the ultrasonic probe 51 of FIGS. 6A to 6C.

### Another Configuration Example of Appearance of Ultrasonic Probe

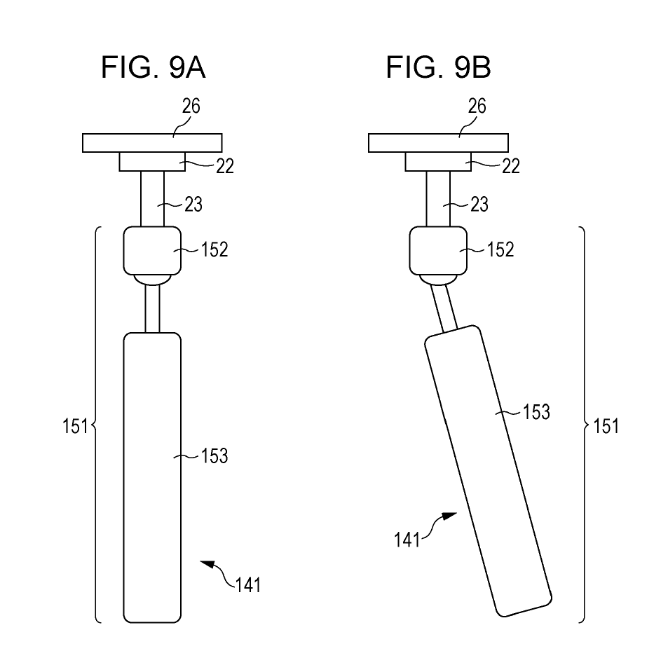

FIGS. 9A and 9B are views showing further still another configuration example of the appearance of the ultrasonic probe according to the present disclosure. FIGS. 9A and 9B are side views from below the joint portion 26 of the ultrasonic probe. In examples of FIGS. 9A and 9B, the description is focused on a handle portion and thereby the illustration of each unit from above the joint portion 26 is omitted.

In examples of FIGS. 9A and 9B, an ultrasonic probe 141 has similarity with the ultrasonic probe 11 of FIGS. 1A to 1C in that the probe (main body) 21, the seat 22, the rotational axis 23, the guide 25, and the joint portion 26 are included. The ultrasonic probe 141 is different from the ultrasonic probe 11 of FIGS. 1A to 1C in that the handle 24 is switched with a handle 151.

In the examples of FIGS. 9A and 9B, the handle 151 is configured to include an auxiliary operation unit 153 having a ball joint 152. As shown in FIG. 9A, the handle 151 is almost vertically provided with respect to an ultrasonic beam of the probe 21, in a similar way to the handle 24 of FIGS. 1A to 1C. Moreover, as shown in FIG. 9B, by using the ball joint 152 of the handle 151, it is possible to make the auxiliary operation unit 153 have an angle with respect to the ultrasonic beam of the probe 21.

The handle 151 may be configured in such a manner that the auxiliary operation unit 153 having the ball joint 152 is added to the handle 24 of FIGS. 1A to 1C. In this case, the auxiliary operation unit 153 having the ball joint 152 may be detachable.

Operability of the handle 24, which the ultrasonic probe 11 of FIGS. 1A to 1C has, is flexible by the rotational axis 23. However, since data acquired by the angle sensor is typically a rotational angle of the probe 21, if the handle 24 is shifted to other hand, it is easy to cause an odd motion. To address this circumstance, by providing the ball joint 152, it is possible to easily perform a maneuver to rotate the probe 21 on the circumference of parts, for example, when parts such as an elbow or a knee are measured.

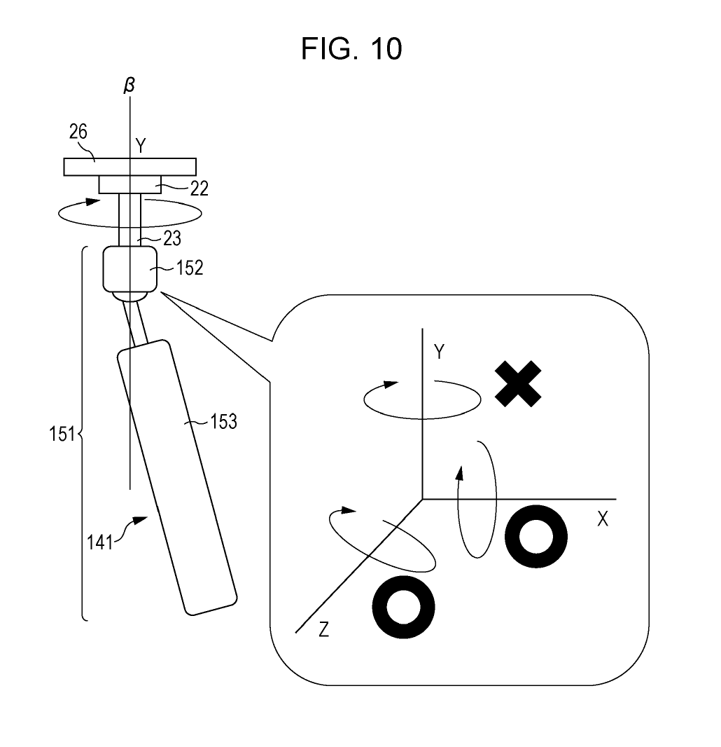

As shown in FIG. 10, since a rotational angle centering on a Y axis, which is represented as the axis β perpendicular to a beam direction, is acquired at the existing rotational axis 23, the ball joint 152, which is positioned below the joint portion 26 of the ultrasonic probe 111, is inhibited from being rotated in the Y axis. Therefore, the ball joint 152 has a degree of freedom in rotation about only X and Z axes.

In examples of FIGS. 9A to 10, the ball joint 152 is used. However, this is not limited to a ball joint, but anything may be used as long as it makes the auxiliary operation unit 153 have an angle with respect to the axis β.

As described above, only by subsequently adding at least one of a plurality of jigs described above to the probe 21, it is possible to stably capture an image around a joint.

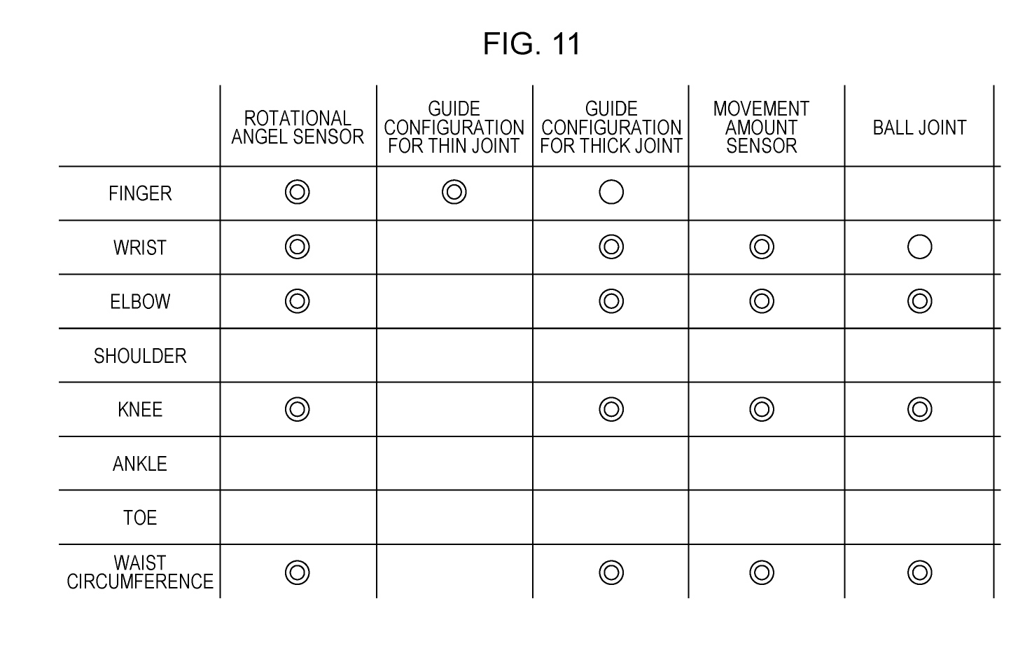

FIG. 11 shows the usability of the above-described configuration elements (jigs) in such a manner that the usability is organized by each diagnosis target. A double-circle mark indicates that a configuration element is useful in a diagnosis target.

The rotational angle sensor is useful in a finger, a wrist, an elbow, a shoulder, a knee, and waist circumference. A configuration having a guide for a thin joint (for example, the guide 25 of FIGS. 1A to 1C) is useful in a finger. That is to say, the guide for a thin joint is a guide specialized for a finger.

A configuration having a guide for a thick joint (for example, the guide 91 of FIGS. 7A to 7C) is useful in a finger, a wrist, an elbow, a knee, and waist circumference. In addition, a circle mark shown in the table cell of a finger indicates that the guide for a thick joint may be used but the guide for a thin joint is more useful than the guide for a thick joint.

The movement amount sensor is useful in a wrist, an elbow, a knee, and a waist circumference. The ball joint is useful in a wrist, an elbow, a knee, and waist circumference. Further, the ball joint is useful in a wrist but may not be necessary.

In the rheumatoid arthritis examination, a toe, a dorsum of the foot, an ankle and the like are included in a diagnosis target but these parts are outside the scope of the present disclosure.

As described above, by only attaching a jig, for example, as a probe supporting apparatus to the existing probe, the above-described things are realized.

Particularly, in a case where a handle having a rotational axis, which an angle sensor is built therein, is attached to a probe in an orthogonal manner, as a jig, it is possible to detect a precise angle of probe which rotates around a cylindrical subject to be imaged.

In a case where a guide is attached as a jig, it is possible to typically transmit and receive an ultrasonic beam to and from the vertical direction with respect to the subject to be imaged and it is easy to rotate the probe around the subject to be imaged. Therefore, it is possible to precisely and easily acquire an ultrasonic image for acquiring a three-dimensional structure of a target portion.

In a case where a ball joint is attached as a jig separately from a rotational axis, it is possible to improve a maneuvering feeling.

Next, a configuration of the image processing system as the ultrasonic processing apparatus which includes the above-described ultrasonic probe will be described below. Any ultrasonic probes having the above-described configurations may be used. However, as an example, the description herein will be made using the ultrasonic probe 111 of FIGS. 8A to 8C.

### Second Embodiment

### Configuration Example of Image Processing System

FIG. 12 is a block diagram showing a configuration example of an image processing system 201 according to the present disclosure.

The image processing system 201 is a system that generates a cross-sectional image representing at least a part of cross sections of the subject to be imaged, using ultrasonic waves, and then displays the generated cross-sectional image. The image processing system 201 is used, for example, as an ultrasonic diagnosis apparatus when an image of cross sections of respective parts, such as an abdominal part of a human, is imaged to examine the captured image.

The image processing system 201 is configured to include the ultrasonic probe 111 of FIGS. 8A to 8C, an image processing device 212, recording devices 213a to 213d, and a display 214.

The ultrasonic probe 111 is configured to include an ultrasonic wave transmission and reception unit 221 and a detection unit 222.

The ultrasonic wave transmission and reception unit 221 is provided, for example, at the distal end of the ultrasonic probe 111 and transmits and receives ultrasonic waves under the control of an ultrasonic wave control unit 251 of the image processing device 212. The ultrasonic wave transmission and reception unit 221 is configured to include an ultrasonic wave generation unit 231 and an ultrasonic wave reception unit 232.

The ultrasonic wave generation unit 231 generates ultrasonic waves under the control of the ultrasonic wave control unit 251. More specifically, for example, the ultrasonic wave generation unit 231 oscillates pulse-shaped ultrasonic waves at a predetermined interval and performs ultrasonic wave scanning.

In an ultrasonic wave scanning method, an arbitrary method can be employed. For example, scanning may be carried out in a radial or parallel manner. When scanning is carried out in a radial manner, a fan-shaped ultrasonic image can be obtained. When scanning is carried out in a parallel manner, a rectangular ultrasonic image can be obtained.

The ultrasonic wave reception unit 232 receives reflective waves of the ultrasonic waves generated by the ultrasonic wave generation unit 231, under the control of the ultrasonic wave control unit 251. Then, the ultrasonic wave reception unit 232 measures the intensity of the received reflective waves and supplies, for example, data representing a time-series measurement result of the intensity of the reflective waves (hereinafter, referred to as ultrasonic wave measurement data) to an ultrasonic image generation unit 252 of the image processing device 212.

The detection unit 222 detects a state of the ultrasonic probe 111 (for example, an angle, a position, or the like). The detection unit 222 is configured to include an angle sensor 241 and the movement amount sensor 121 of FIGS. 8A to 8C. The angle sensor 241 detects, for example, a rotational angle of the ultrasonic probe 111. The movement amount sensor 121 detects a movement amount of the ultrasonic probe 111. In addition, as described in FIG. 12, the detection unit 222 may be configured to include an angular velocity sensor 242 configured by a gyro or the like.

Each sensor of the detection unit 222 supplies sensor data, which represents a detection result, to a sensor information acquisition unit 253 of the image processing device 212.

The image processing device 212 performs processes of generating a cross-sectional image of a subject to be imaged and displaying the generated cross-sectional image on the display 214. The image processing device 212 is configured to include the ultrasonic wave control unit 251, the ultrasonic image generation unit 252, the sensor information acquisition unit 253, a probe state detection unit 254, a cross-sectional image generation unit 255, a display control unit 256, and a simplified display image generation unit 257.

The ultrasonic wave control unit 251 controls the ultrasonic wave generation unit 231 and the ultrasonic wave reception unit 232, and controls transmission and reception of ultrasonic waves of the ultrasonic probe 111.

The ultrasonic image generation unit 252 generates an ultrasonic image based on ultrasonic wave measurement data supplied from the ultrasonic wave reception unit 232.

Therefore, processes of generating ultrasonic waves, receiving reflective waves thereof and generating an ultrasonic image based on the received reflective waves, that is, imaging an ultrasonic image are performed by the ultrasonic wave generation unit 231, the ultrasonic wave reception unit 232, the ultrasonic wave control unit 251, and the ultrasonic image generation unit 252.

The ultrasonic image generation unit 252 stores ultrasonic image data representing the generated ultrasonic image in the recording device 213a.

The sensor information acquisition unit 253 acquires information representing a state of the ultrasonic probe 111, such as an angle or a position thereof. Specifically, the sensor information acquisition unit 253 performs sampling of a detection value of each sensor at a predetermined interval, based on sensor data supplied from each sensor of the ultrasonic probe 111. Then, the sensor information acquisition unit 253 stores the sampled detection value of each sensor and a time when sampling the detection value of each sensor, as sensor information, in the recording device 213b.

The probe state detection unit 254 detects a state of the ultrasonic probe 111 at the time of imaging an ultrasonic image, based on the sensor information stored in the recording device 213b, and supplies the detection result to the cross-sectional image generation unit 255. The probe state detection unit 254 selectively uses information for detecting a state of the ultrasonic probe 111, among the sensor information stored in the recording device 213b.

The cross-sectional image generation unit 255 performs volume interpolation by arranging an ultrasonic image in a display region in a three-dimensional manner and then generates a cross-sectional image (three-dimensional volume data) of a subject to be imaged, based on an ultrasonic image stored in the recording device 213a and a state of the ultrasonic probe 111 at the time of imaging an ultrasonic image. The cross-sectional image generation unit 255 stores cross-sectional image data representing the generated cross-sectional image in the recording device 213c.

The display control unit 256 displays the cross-sectional image of a subject to be imaged on the display 214, based on the cross-sectional image data stored in the recording device 213c. The display control unit 256 displays a simplified display image group in which a plurality of simplified display images (preview images) are arranged in a three-dimensional manner so as to be bent in a three-dimensional manner, on the display 214, based on the simplified display image group data stored in the recording device 213d.

When one of the plurality of simplified display images in the displayed simplified display image group is selected, the display control unit 256 respectively reads an ultrasonic image corresponding to the simplified display images and information on the ultrasonic image from the recording device 213a and the recording device 213b and then displays the ultrasonic image and information of the ultrasonic image on the same screen.

That is to say, the simplified display image group is displayed as an index for viewing an ultrasonic image, on the display 214.

Interlocking with the rotating operation of the ultrasonic probe 111, the simplified display image generation unit 257 generates a simplified display image group in which a plurality of simplified display images corresponding to a plurality of ultrasonic images are arranged so as to be bent in a three-dimensional manner by using plural items of ultrasonic image data stored in the recording device 213a. The simplified display image generation unit 257 stores simplified display image group representing the generated simplified display image group in the recording device 213d.

For example, interlocking with the rotating operation of the ultrasonic probe 111, the simplified display image generation unit 257 arranges the ultrasonic image data stored in the recording device 213a in the display region. At this time, the ultrasonic image data is arranged in the display region such that the ultrasonic image data is arranged and displayed in a three-dimensional manner or is bent in a three-dimensional manner to be displayed.

Then, the simplified display image generation unit 257 generates a simplified display image group by generating a plurality of simplified display images corresponding to the plurality of ultrasonic images which are arranged in the display region, as described above, interlocking with the rotating operation of the ultrasonic probe 111. In this way, the display control unit 256 arranges the plurality of simplified display images generated by the simplified display image generation unit 257 at a position interlocking with a rotating operation of the probe to display the plurality of simplified display images on a display screen.

If it is considered that an ultrasonic image (simplified display image) is recognized by a user in a spatial state by the ultrasonic image being arranging in a display region in a three-dimensional manner to be displayed or being bent in a three-dimensional manner to be displayed, the display region may also be defined as space (or virtual space).

The recording device 213a is configured by, for example, a scene memory and stores ultrasonic image data representing the ultrasonic image generated by the ultrasonic image generation unit 252. The recording device 213b stores a detection value of each sensor and a time when sampling the detection value of each sensor, as sensor information.

The recording device 213c stores cross-sectional image data representing the cross-sectional image generated by the cross-sectional image generation unit 255. The recording device 213d stores simplified display image group data representing the simplified display image group generated by the simplified display image generation unit 257.

The display 214 displays an image under the control of the display control unit 256.

In an example of FIG. 12, a case where the recording devices 213a to 213d are provided separately from the image processing device 212 is exemplified. However, the recording devices 213a to 213d may be provided in the image processing device 212.

Imaging Processes

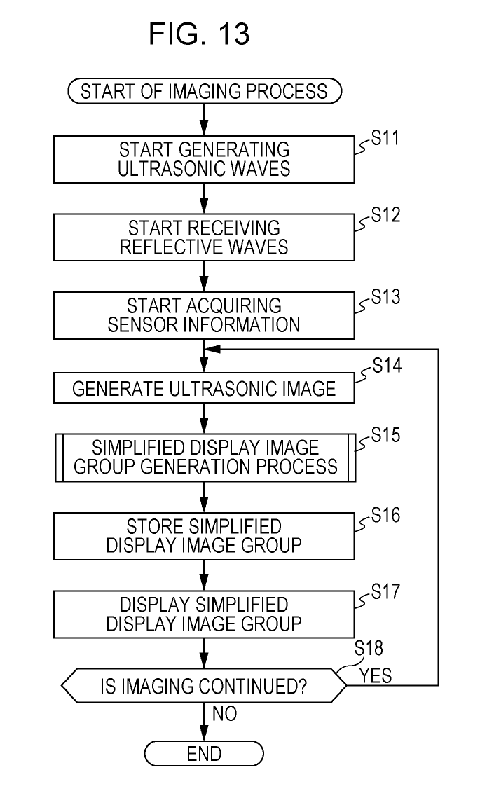

Next, with reference to a flowchart of FIG. 13, imaging processes executed by the image processing system 201 will be described. These processes are started, for example, when a starting command for imaging is inputted by an operation unit (not shown) of the image processing system 201.

Hereinafter, a case where a cross section of a human's joint is imaged by using the image processing system 201 is exemplified. In this case, for example, in order to image a cross section of joints such as joints of a finger or joints of an elbow, a person to be imaged allows the ultrasonic probe 111 to almost vertically come into contact with his or her joint and then to circle around the joint, as shown in FIGS. 2 to 5.

The ultrasonic probe 111 can almost vertically come into contact with a joint to circle around the joint by the above-described jigs, with ease.

The ultrasonic probe 111 may be operated by a person other than a person to be imaged or may be remotely operated by a robot arm or the like.

In Step S11, the ultrasonic wave generation unit 231 starts generating ultrasonic waves under the control of the ultrasonic wave control unit 251. For example, the ultrasonic wave generation unit 231 scans ultrasonic waves in a predetermined direction while oscillating pulse-shaped ultrasonic waves at a predetermined interval.

In Step S12, the ultrasonic wave reception unit 232 starts receiving reflective waves of the ultrasonic waves generated by the ultrasonic wave generation unit 231 under the control of the ultrasonic wave control unit 251. Then, the ultrasonic wave reception unit 232 measures the intensity of the received reflective waves and supplies ultrasonic wave measurement data representing the measurement result to the ultrasonic image generation unit 252.

In Step S13, the sensor information acquisition unit 253 starts acquiring sensor information. Specifically, the sensor information acquisition unit 253 performs sampling of a detection value of each sensor at a predetermined interval, based on sensor data supplied from each sensor of the ultrasonic probe 111. Then, the sensor information acquisition unit 253 stores the sampled detection value of each sensor and a time when sampling the detection value of each sensor, as sensor information, in the recording device 213b.

In Step S14, the ultrasonic image generation unit 252 generates an ultrasonic image based on the ultrasonic wave measurement data supplied from the ultrasonic wave reception unit 232. That is, the ultrasonic image generation unit 252 generates a second-dimensional ultrasonic image representing an internal cross section of a joint of a person to be imaged near a position where the joint comes into contact with the ultrasonic probe 111. The ultrasonic image generation unit 252 stores ultrasonic image data representing the generated ultrasonic image and an imaging time in the recording device 213a.

The method for generating an ultrasonic image is not limited to a specific method and an arbitrary method may be employed.

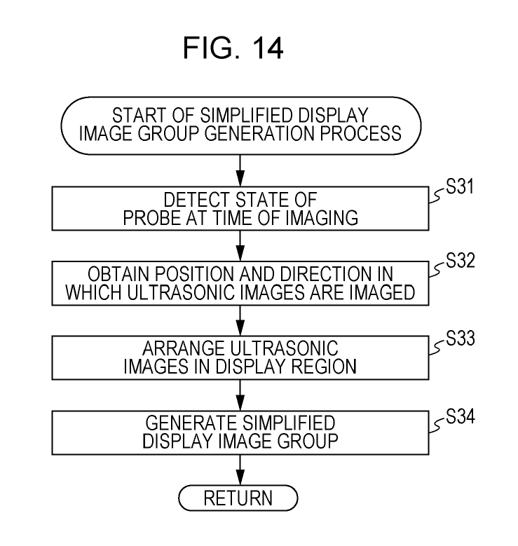

In Step S15, the simplified display image generation unit 257 executes a simplified display image group generation process. Although details of the simplified display image group generation process will be described later with reference to FIG. 14, a simplified display image group in which a plurality of simplified display images corresponding to a plurality of ultrasonic images are arranged to be bent in a three-dimensional manner is generated by the process of Step S15.

In Step S16, the simplified display image generation unit 257 stores simplified display image group data representing the generated simplified display image group in the recording device 213d.

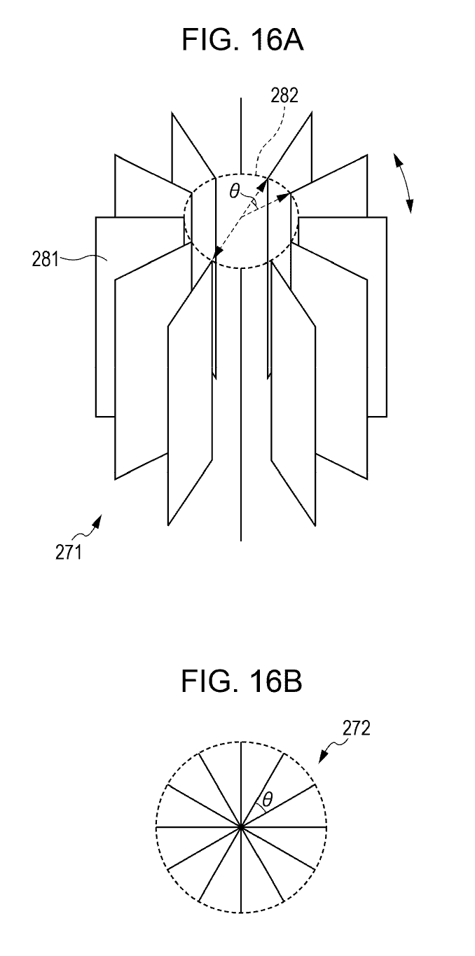

In Step S17, the image processing system 201 displays the simplified display image group. Specifically, the display control unit 256 reads the simplified display image group data from the recording device 213d. Then, the display control unit 256 displays the simplified display image group based on the read simplified display image group data, on the display 214. In this way, the simplified display image group described later in FIGS. 16A and 16B is displayed on the display 214.

In Step S18, the image processing system 201 determines whether or not to continue imaging. When it is determined to continue imaging, the process returns to Step S14.

Thereafter, until it is determined not to continue imaging in Step S18, processes of Steps S14 to S18 are repeatedly executed. In other words, processes of imaging an ultrasonic image, generating a simplified display image, and displaying the simplified display image are continuously preformed.

An interval of imaging an ultrasonic image and generating a simplified display image or a cross-sectional image is determined, for example, with consideration for system processing capacity, capacity of the recording devices 213a to 213d, or the like. Moreover, when the image processing system 201 is driven by a battery, battery capacity or the like may be considered.

On the other hand, in Step S18, for example, when an ending command for imaging is inputted by the operation unit (not shown) of the image processing system 201, the image processing system 201 determines not to continue imaging and then the imaging process is ended.

Details of Simplified Display Image Group Generation Process

Next, with reference to a flowchart of FIG. 14, details of the simplified display image group generation process in Step S15 of FIG. 13 will be described.

In Step S31, the probe state detection unit 254 detects a state of the ultrasonic probe 111 at the time of imaging, based on the sensor information stored in the recording device 213b.

Specifically, the probe state detection unit 254 obtains variation (trajectory) in a position and a direction (angle) of the ultrasonic probe 111 up to the present time, based on the detection result of the angle sensor 241 and the movement amount sensor 121.

As described above, a detection value of each sensor is discretely obtained at a predetermined sampling interval. Herein, the probe state detection unit 254 obtains variation in a position and an angle of the ultrasonic probe 111 by interpolating the detection value of each sensor, as necessary.

A method for interpolation, which is used at this time, is not limited to a specific method but, for example, it is assumed that the motion of the ultrasonic probe 111 at the time of imaging is smooth and then linear interpolation, spline interpolation or the like is performed.

Then, the probe state detection unit 254 detects a position and angle of the ultrasonic probe 111 when ultrasonic wave generation and reflective wave reception are performed in order to image a latest ultrasonic image, based on the variation in a position and angle of the ultrasonic probe 111.

Thereafter, the probe state detection unit 254 supplies the detection result of the state of the ultrasonic probe 111 at the time of imaging, to the simplified display image generation unit 257.

In Step S32, the simplified display image generation unit 257 obtains a position and a direction (angle) in which ultrasonic images are imaged. Specifically, the simplified display image generation unit 257 calculates a position (imaging position) and an angle (imaging angle) in which a latest ultrasonic image is imaged, based on the position and angle of the ultrasonic probe 111 at the time of imaging the latest ultrasonic image. The imaging start position and angle of the ultrasonic probe 111 are determined in advance and the relationship between the position and angle of the ultrasonic probe 111 and the stereotactic position (imaging position and imaging angle) of the ultrasonic images is obtained in advance.

In Step S33, the simplified display image generation unit 257 arranges ultrasonic images in a display region. Specifically, for example, the simplified display image generation unit 257 reads a latest ultrasonic image from the recording device 213a. Then, the simplified display image generation unit 257 arranges the latest ultrasonic image in the display region (space) where the ultrasonic images before one frame are arranged in a three-dimensional manner, based on the imaging position and imaging angle of the latest ultrasonic image. The relative positional relationship of each ultrasonic image can be obtained by using the imaging position and imaging angle of each ultrasonic image.

The simplified display image generation unit 257 adjusts a position where an ultrasonic image is arranged, based on information on ultrasonic images.

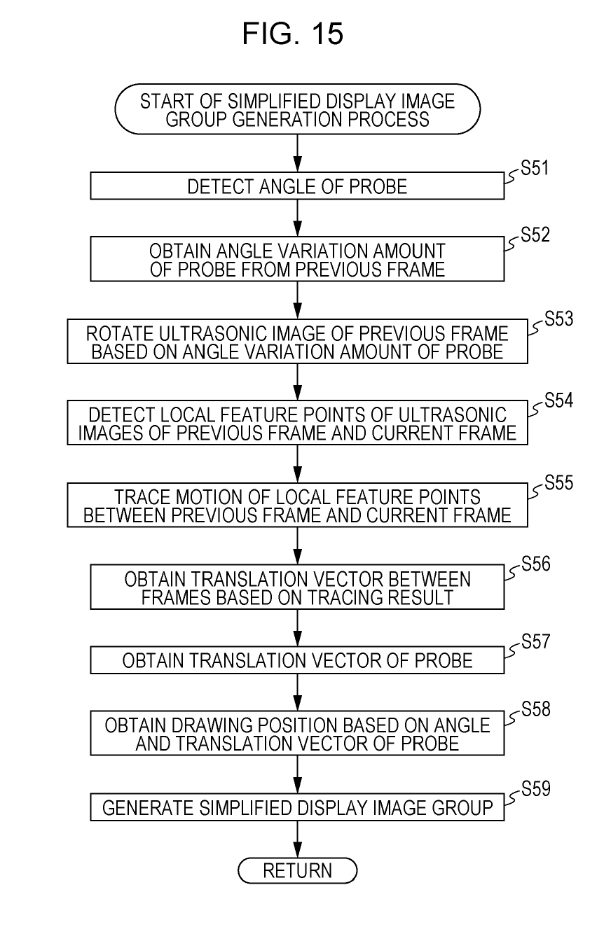

For example, the simplified display image generation unit 257 detects a feature point of the latest ultrasonic image. Then, the simplified display image generation unit 257 adjusts a position where each ultrasonic image is arranged, by tracing the trajectory of feature points of ultrasonic images up until now. Specific examples thereof will be described later, for example, with reference to FIG. 15.

For example, a sensor is not generally good at detecting motion in a translation direction and thus the detection error tends to be large. Therefore, when only sensor information is used, there is a case where the accuracy of aligning ultrasonic images deteriorates. To address this circumstance, by using not only sensor information but also information on ultrasonic images, the accuracy of aligning ultrasonic images improves.

In contrast, since an ultrasonic image generally contains a lot of noise, it is difficult to perform the alignment of ultrasonic images with a good accuracy, by using only information on ultrasonic images. To address this circumstance, by using not only information on ultrasonic images but also sensor information, the accuracy of aligning ultrasonic images improves.

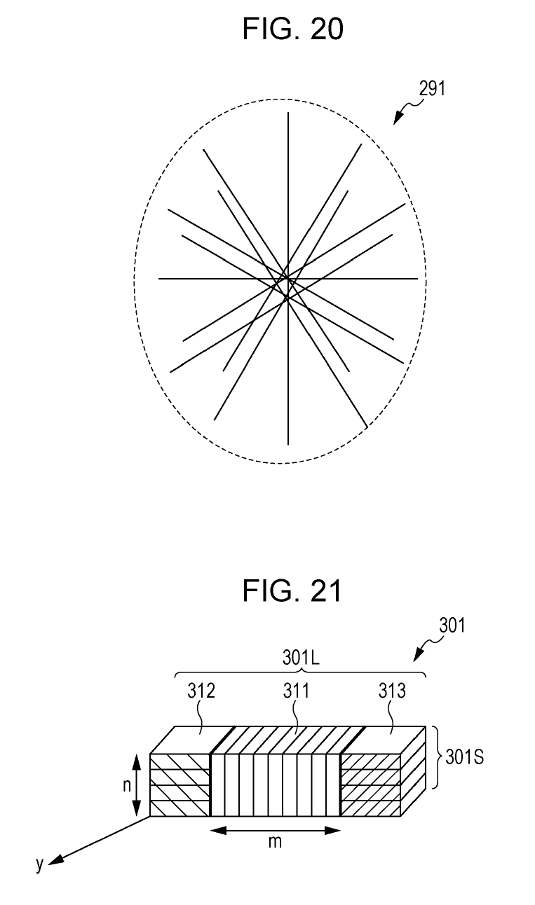

As in the case of imaging a finger joint, if ultrasonic images are arranged on a three-dimensional region of the display region and the arrangement thereof is a completely round circle when seen from the top (refer to FIG. 16B described later), it is possible to perform the arrangement thereof by using only angle information detected by the angle sensor 241. On the other hand, as in the case of imaging a wrist, an elbow, a waist or the like, if ultrasonic images are arranged on a three-dimensional region of the display region and the arrangement thereof is an elliptical shape when seen from the top (refer to FIG. 20 described later), information on movement amounts from the movement amount sensor 121 is also necessary.

However, even in a case where ultrasonic images are arranged on a three-dimensional region of the display region and the arrangement thereof is an elliptical shape when seen from the top, if assuming the uniform motion, it is possible to perform the arrangement thereof by using only angle information detected by the angle sensor 241. That is to say, in this case, it is necessary to rotate the ultrasonic probe 111 around a diagnosis target object at almost constant speed.

In Step S34, the simplified display image generation unit 257 generates simplified display image group. That is, the simplified display image group 257 generates a simplified display image corresponding to the arrangement position of ultrasonic images which are arranged at the display region in a three-dimensional manner, from the ultrasonic images. Then, the simplified display image generation unit 257 generates a simplified display image group in which a plurality of simplified display images, which correspond to a position where an ultrasonic image is arranged, are arranged in a three-dimensional manner, that is, in which the plurality of simplified display images are bent in a three-dimensional manner.

In the process of Step S34, if the ultrasonic probe 111 has not circled around a joint yet, a simplified display image group representing a progress up to the range where an ultrasonic image is imaged may be generated. Alternatively, a simplified display image group may be generated after all imaging has been completed.

### Another Example of Simplified Display Image Group Generation Process

With reference to a flowchart of FIG. 15, a modification example of the simplified display image group generation process in Step S15 of FIG. 13 will be described in detail.

For example, a description will be made of a case where an ultrasonic image is imaged in such a manner that the guide 91 of the ultrasonic probe 111 vertically comes into contact with a joint and then the ultrasonic probe 111 is horizontally circled around the joint by allowing the ultrasonic probe 111 to rotate about the rotational axis 23 as shown in FIGS. 2 to 5 described above. Moreover, for example, by setting a sufficiently fast frame rate or moving the ultrasonic probe 111 slowly, an ultrasonic image is imaged such that imaging ranges of adjacent frames are overlapped.

In Step S51, the probe state detection unit 254 detects an angle of the ultrasonic probe 111 at the time of imaging, based on the detection result of the angle sensor 241, which is stored in the recording device 213b.

In Step S52, the probe state detection unit 254 obtains an angle variation amount of the ultrasonic probe 111 from the previous frame, based on the detection result of the angle of the ultrasonic probe 111.

The probe state detection unit 254 supplies information representing the obtained angle variation amount of the ultrasonic probe 111 to the simplified display image generation unit 257.

In Step S53, the simplified display image generation unit 257 rotates the ultrasonic image of the previous frame, based on the angle variation amount of the ultrasonic probe 111.

In Step S54, the simplified display image generation unit 257 detects local feature points of the ultrasonic images of the previous frame and the current frame. More specifically, the simplified display image generation unit 257 detects local feature points of the rotated image of the previous frame and the local feature points of the ultrasonic image of the current frame.

Any kind of local feature point and any detection method can be employed. For example, the Harris Corner Detector, which is resilient against the deformation of a subject to be imaged and suitable for soft human tissues, is used as the local feature point.

In Step S55, the simplified display image generation unit 257 traces the motion of the local feature points between the previous frame and the current frame. More specifically, the simplified display image generation unit 257 traces the motion of the local feature points between the rotated image of the previous frame and the ultrasonic image of the current frame.

Any method can be employed as the method of tracing the motion of the local feature points. For example, an Optical Flow Lucas-Kanade method, which is resilient against the deformation of a subject to be imaged and suitable for soft human tissues, is used.

In Step S56, the simplified display image generation unit 257 obtains a translation vector between the frames based on the tracing result.

In Step S57, the simplified display image generation unit 257 obtains a translation vector of the ultrasonic probe 111. Specifically, the simplified display image generation unit 257 obtains an inverse vector inverted from the translation vector T obtained in the process of Step S56 as the translation vector of the ultrasonic probe 111. The translation vector expresses the motion of the ultrasonic probe 111 in the translation direction from the imaging time of the previous ultrasonic image to the imaging time of the next ultrasonic image UI.

In Step S58, the simplified display image generation unit 257 obtains the drawing position based on the angle and the translation vector of the ultrasonic probe 111. Specifically, the simplified display image generation unit 257 obtains the drawing position of the ultrasonic image of the current frame in the three-dimensional virtual space, where the ultrasonic images up to the immediate previous frame are arranged, based on the angle of the ultrasonic probe 111 detected by the angle sensor 241 and the translation vector of the detected ultrasonic probe 111.

For example, the simplified display image generation unit 257 obtains the relative variation amount of drawing position between the ultrasonic images of the immediate previous frame and the current frame, based on the angle and the translation vector of the ultrasonic probe 111. Then, the simplified display image generation unit 257 obtains the drawing position of the ultrasonic image of the current frame in the display region based on the obtained variation amount.

In Step S59, the simplified display image generation unit 257 generates the simplified display image group. That is, the simplified display image generation unit 257 generates the simplified display image group by arranging the simplified display image corresponding to the ultrasonic image of the current frame at the obtained drawing position and synthesizing the simplified display image with the simplified display image up to the current frame.

Thereafter, the simplified display image group generation process is ended.

In Steps S15 to S17 of FIG. 13 and FIGS. 14 and 15 as described above, the example has been described in which the simplified display image group was generated, stored and displayed. However, information from the probe state detection unit 254 is also supplied to the cross-sectional image generation unit 255. Therefore, it is possible to generate a cross-sectional image instead of the simplified display image group.

In a case of the cross-sectional image, the cross-sectional image generation unit 255 can generate the cross-sectional image (3D volume data) by performing volume interpolation using the ultrasonic images arranged in the display region in the three-dimensional manner.

In the above-described description, the example has been described in which the simplified display image group is generated using the ultrasonic image but the method of generating the simplified display image group is not limited thereto. For example, the cross-sectional image (3D volume data) is generated by performing volume interpolation using the ultrasonic images arranged in the display region in the three-dimensional manner as described above and then the simplified display image group may be generated. In this way, it is possible to browse the simplified display image group from an arbitrary cross section.

Both of the simplified display image group and the cross-sectional image are generated and then both images may be arranged and displayed on the screen.

When these obtained sequential ultrasonic images are stored in the recording device 214a that is a scene memory, a position in the memory corresponds to an angle. Therefore, it is possible to search the ultrasonic image of the direction where the user wants to watch while moving in the memory using a slider. In this case, for example, it is preferable that the simplified display image group described below be displayed as an index.

In the above description, the example has been described in which the cross-sectional image is generated and displayed in real time while imaging the ultrasonic image. However, all ultrasonic images are firstly imaged and thereafter the cross-sectional image may be generated and displayed.

The imaging of the ultrasonic image and the acquisition of the sensor information may or may not be synchronized. When the imaging of the ultrasonic image and the acquisition of the sensor information are not synchronized, it is preferable to record the time at which the ultrasonic images are imaged and the time at which the sensor information is acquired such that the correspondent relationship can be recognized later.

The kinds of sensors provided on the above-described ultrasonic probe 111 are merely examples thereof. As necessary, the kinds of sensor may be added or different kinds of sensors can be used.

In the present disclosure, for example, it is possible to generate the simplified display image group from the ultrasonic images by using only sensor information without using ultrasonic image information.

### Third Embodiment

### Example of Simplified Display Image Group

FIGS. 16A and 16B shows an example of the simplified display image group displayed in Step S17 of FIG. 13 described above. FIG. 16A shows a simplified display image group 271 displayed on the display 214. FIG. 16B is a view showing an arrangement image 272 representing the arrangement of the simplified display image group 271 when seen from the top, that is, a view of which the ultrasonic images, that is the source of the simplified display image group of FIG. 16A, are arranged in the display region when seen from the top. The arrangement image 272 may also be displayed with the simplified display image group 271.

The simplified display image group 271 is configured by a plurality of simplified display images 281 corresponding to the plurality of ultrasonic images. In examples of FIGS. 16A and 16B, the simplified display image group 271 is configured by 12 sheets of the simplified display images 281 but the number of configuration sheets is not limited to 12.

The plurality of simplified display images 281 is distorted in the three-dimensional manner to be displayed such that the plurality of simplified display images 281 surround a circle 282 representing the diagnosis target object.

The position of the simplified display image 281 is obtained based on angle information detected by the angle sensor 241 of the ultrasonic probe 111. That is to say, the plurality of simplified display images 281 are generated and displayed by interlocking with the rotating operation (the angle obtained from the angle sensor 241) of the ultrasonic probe 111 (at a position interlocking therewith).

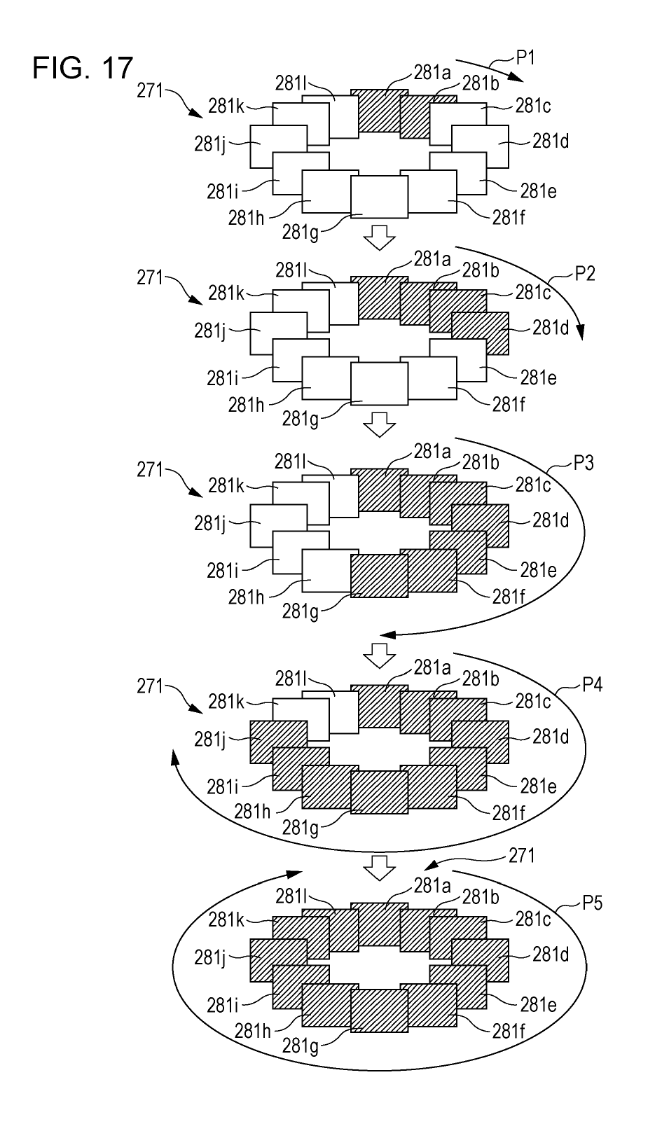

FIG. 17 is a view showing the interlocking with the rotating operation of the ultrasonic probe 111 and the simplified display image group 271.

In examples of FIG. 17, simplified display images 281a to 281l configuring the simplified display image group 271 are shown. Herein, arrows P1 to P5 represent the rotating operation of the ultrasonic probe 111 and hatched images among the simplified display images 281a to 281l represent the images which are generated and displayed by interlocking with the rotating operation of the ultrasonic probe 111. In examples of FIG. 17, although images are only hatched, practically, when the simplified display images 281a to 281l are displayed, the generated preview images are displayed.

Firstly, a person to be imaged allows the ultrasonic probe 111 to perform the rotating operation up to the position represented by the arrow P1. Therefore, interlocking with the rotating operation, the simplified display images 281a and 281b are generated and displayed at each position interlocking therewith. Then, the person to be imaged allows the ultrasonic probe 111 to perform the rotating operation up to the position represented by the arrow P2. Therefore, interlocking with the rotating operation, the simplified display images 281c and 281d are generated and displayed at each position interlocking therewith.

The person to be imaged allows the ultrasonic probe 111 to perform the rotating operation up to the position represented by the arrow P3. Therefore, interlocking with the rotating operation, the simplified display images 281e to 281g are generated and displayed at each position interlocking therewith. Then, the person to be imaged allows the ultrasonic probe 111 to perform the rotating operation up to the position represented by the arrow P4. Therefore, interlocking with the rotating operation, the simplified display images 281h to 281j are generated and displayed at each position interlocking therewith.

Then, finally, the person to be imaged allows the ultrasonic probe 111 to perform the rotating operation up to the position represented by the arrow P5. Therefore, interlocking with the rotating operation, the simplified display images 281k and 281l are generated and displayed at each position interlocking therewith.

In this way, the simplified display images 281a to 281l configuring the simplified display image group 271 are generated and displayed by interlocking with the rotating operation of the ultrasonic probe 111.

In examples of FIG. 17, images not to be hatched (that is, images which have not been generated yet) are also displayed, but the images to be hatched may be non-displayed or may be displayed in advance.

Returning to FIGS. 16A and 16B, the gap between the simplified display images 281, which is represented by an angle θ between the simplified display images 281, may be set in advance but may be interlocked with the rotating operation of the ultrasonic probe 111. For example, when the angular velocity, which is an angle variation amount detected from the angle sensor 241 of the ultrasonic probe 111, is small, the gap between the simplified display images 281 may be set to be narrow. When the angle velocity is large, the gap between the simplified display images 281 may be set to be wide. The angular velocity information may be obtained from the angle information detected from the angle sensor 241 of the ultrasonic probe 111 and the time information or may be obtained from the angular velocity sensor 242.

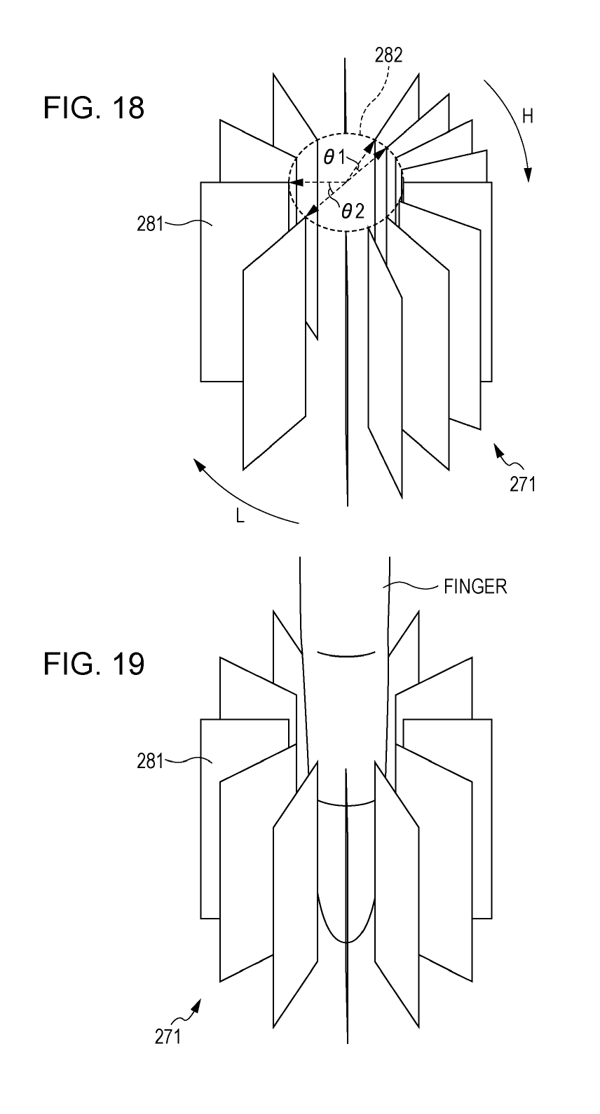

As the description with reference to FIG. 18, for example, in the range represented by the arrow H, the angular velocity is small, that is, the ultrasonic probe 111 is subjected to the rotating operation slowly. In this case, in the range represented by the arrow H, as represented by an angle θ1 between the simplified display images 281, the interval of generating (displaying) the simplified display images 281 becomes narrow.

On the other hand, in the range represented by the arrow L, the angular velocity is large, that is, the ultrasonic probe 111 is subjected to the rotating operation fast. In this case, in the range represented by the arrow L, as represented by an angle θ2 between the simplified display images 281, the interval of generating (displaying) the simplified display images 281 becomes wide.

In this way, it is recognized that the position is a focus position of the subject to be imaged or a non-focus position thereof, in accordance with the gap between the simplified display images 281. Particularly, when the interval of the simplified display images 281 is narrow (the angular velocity is small), the subject to be imaged focuses on the portion where the interval of the simplified display images 281 is narrow and thus it is recognized that the portion is an important position.

In an example of FIG. 18, the example in which the gap of simplified display images is changed in accordance with the rotating operation (angle velocity) of the ultrasonic probe 111 has been described, but the size of simplified display images may be changed in accordance with the obtaining operation (angle velocity) of the ultrasonic probe 111.

For example, the simplified display images 281 in the range where the angular velocity shown in the arrow H is small may be displayed large. On the other hand, the simplified display images 281 in the range where the angular velocity shown in the arrow L is large may be displayed small.

The size of the circle 282, which the simplified display images 281 surround, is displayed interlocking with the size of the diagnosis target (that is, the length of the circumference of a joint or the like).

By displaying images as described above, it is possible to easily and intuitively check the diagnosis target position.

In examples of FIGS. 16A to 18, only simplified display image group is displayed. However, for example, an image corresponding to the diagnosis target and the simplified display image group may be displayed in an overlapping manner. That is, in the example of FIG. 19, an image corresponding to the diagnosis target (for example, a finger image) is displayed at the position of the circle 282 shown in FIGS. 16A and 16B and then the simplified display image group 271 is overlapped on the image so as to be displayed. As displayed above, it is possible to more intuitively check the diagnosis target position.

For example, when one simplified display image 281 is selected, an ultrasonic image corresponding to the selected simplified display image 281 is read from the recording device 213a and is displayed on the same screen. In this case, information on the ultrasonic image corresponding to the selected simplified display image 281 is further read from the recording device 213b and may be displayed at the same time.

In this way, it is possible to perform the follow-up observation at the same angle as the angle in which is used in browsing at the time of the previous recording.

In examples of FIGS. 16A and 16B, as shown in an arrangement image 272 of FIG. 16B, the diagnosis target object in a case where the cross section of a finger joint or the like is substantially round circle is shown.

For example, the cross section of an elbow, a wrist or the like becomes elliptical as shown in an arrangement image 291 of FIG. 20. In this case, the simplified display image group and the cross-sectional image are generated based on the movement amount detected from the movement amount sensor 121, in addition to the angle detected from the angle sensor 241 of the ultrasonic probe.

That is to say, it is possible to reconstruct a precise shape matching a structure of human body (for example, an elliptical shape) in addition to a completely round circle.

In the above-described ultrasonic probe 111, the example in which the simplified display image group is generated using the angle sensor 241 and the movement amount sensor 121 has been described. However, although the accuracy falls down a little, the angular velocity sensor 242 may be used instead of the angle sensor 241. In addition, it is possible to obtain the movement amount by using an ultrasonic probe described next, without using the movement amount sensor 121.

### Fourth Embodiment

### Another Configuration Example of Ultrasonic Probe

FIG. 21 is a view showing a configuration example of the probe according to the present disclosure.

An ultrasonic probe 301 shown in FIG. 21 is configured to include an A array oscillator 311, a B array oscillator 312 and a C array oscillator 313. In the example of FIG. 21, only array oscillators configuring the ultrasonic probe 301 are shown, but these array oscillator are generally provided in a case such as the above-described ultrasonic probe 11 of FIGS. 1A to 1C.

The A array oscillator 311 is, for example, the basically same one-dimensional array oscillator as the oscillator which the probe 21 of FIGS. 1A to 1C has. The B array oscillator 312 and the C array oscillator 313 is connected to both ends (both of left and right ends in the drawing) of the short side of the A array oscillator 311 such that the arrangement direction of each oscillator of the A array oscillator 311 is orthogonal to the arrangement direction of each oscillator of the B array oscillator 312 and the C array oscillator 313.

That is, each oscillator of the A array oscillator 311 is arranged along a long side 301L of the ultrasonic probe 301 in a similar way to the oscillator or the like in the probe 21 of FIGS. 1A to 1C. On the other hand, each oscillator of the B array oscillator 312 and the C array oscillator 313 is arranged along a short side 301S of the ultrasonic probe 301.

In this way, the B array oscillator 312 and the C array oscillator 313 are arranged to be oriented in the tangent line direction of the rotation of the ultrasonic probe 301. Therefore, it is possible to easily perform motion detection and rotation detection which are described later.

Herein, the length of the long side 301L of the ultrasonic probe 301 is the length including (the length of the long side of each oscillator of the B array oscillator 312)+(the length of the arrangement direction of the A array oscillator 311)+(the length of the long side of each oscillator of the C array oscillator 313). The length of the short side 301S of the ultrasonic probe 301 is the length including (the length of the long side of each oscillator of the A array oscillator 311) and (the lengths of the arrangement directions of the B array oscillator 312 and the C array oscillator 313).

The lengths of the arrangement directions of the B array oscillator 312 and the C array oscillator 313 are shorter than the length of the arrangement direction of the A array oscillator 311. The shape of the oscillator configuring each array oscillator is generally considered to be same. That is to say, the number (n) of oscillators, which are arranged in the B array oscillator 312 and the C array oscillator 313, is less than the number (m) of oscillators, which are arranged in the A array oscillator 311.

As described above, the B array oscillator 312 and the C array oscillator 313 are different from the A array oscillator 311 only in the arrangement number and the direction in which oscillators are arranged in the ultrasonic probe 301. Other configurations thereof are generally same as the A array oscillator 311.

In the example of FIG. 21, a case where each number of oscillators, which are arranged in the B array oscillator 312 and the C array oscillator 313, is the same number n is shown. However, each number of oscillators, which are arranged in the B array oscillator 312 and the C array oscillator 313, may be different to each other as long as it is less than the number of oscillators of the A array oscillator 311.

The physical configurations or characteristics of the oscillators, which configures the ultrasonic probe 301, such as types, physical properties or fillers are not limited.

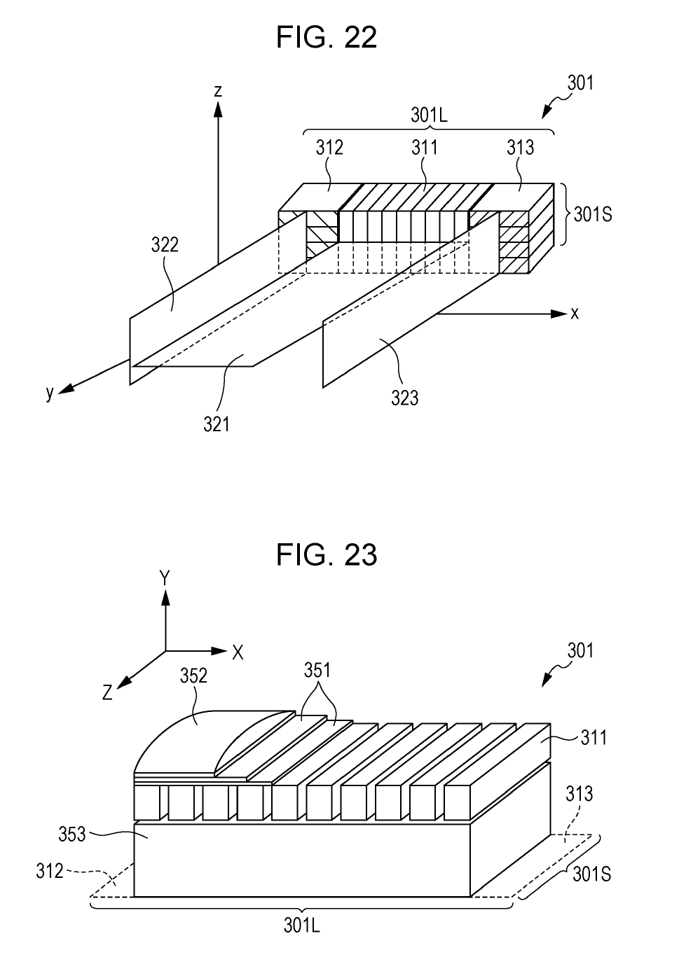

In the ultrasonic probe 301 configured as described above, as shown in FIG. 22, it is possible to reconstruct images in three scanning surfaces.

### Example of Image Surface of Array Oscillator

FIG. 22 is a view showing an image surface of each array oscillator.

In the example of FIG. 22, in the drawing, the right direction is the forward direction of the x axis, the upper direction is the forward direction of the z axis direction, and the lower left front direction is the forward direction of the y axis. An A plane 321, a B plane 322, and a C plane 323 are shown so as to be perpendicular to a zx plane which is formed by the x axis in the direction along the long side 301L of the ultrasonic probe 301 (the arrangement direction of the array oscillator 311) and the z axis in the direction along the short side 301S of the ultrasonic probe 301 (the arrangement direction of the B array oscillator 312 and the C array oscillator 313).

That is, the A plane 321 is disposed at the center of the long side of oscillators which are arranged in the A array oscillator 311. The A plane 321 is a scanning surface parallel to an xy plane and an image surface which is reconstructed in a scanning surface perpendicular to the zx plane.

The B plane 322 is disposed at the center of the long side of oscillators which are arranged in the B array oscillator 312. The B plane 322 is a scanning surface parallel to an yz plane and an image surface which is reconstructed in a scanning surface perpendicular to the zx plane.

The C plane 323 is disposed at the center of the long side of oscillators which are arranged in the C array oscillator 313. The C plane 323 is a scanning surface parallel to an yz plane and an image surface which is reconstructed in a scanning surface perpendicular to an xz plane.

That is, the B plane 322 and the C plane 323 are planes parallel to each other and planes respectively perpendicular to the A plane 321.

As described above, in the ultrasonic probe 301, the A array oscillator 311, the B array oscillator 312 and the C array oscillator 313 are provided such that the B plane 322 and the C plane 323 are planes parallel to each other and planes respectively perpendicular to the A plane 321.

Hereinafter, the ultrasonic probe 301, which is configured to have the three scanning surface as described above, is also referred to as a three plane probe.

Acoustic Lens in Probe

FIG. 23 shows an internal structure of the side to come into contact with the subject to be imaged of the A array oscillator 311 in the ultrasonic probe 301. In the example of FIG. 23, in the drawing, the upper direction is the forward direction of the y axis and the side where the ultrasonic probe 301 comes into contact with the subject to be imaged. In the drawing, the right direction is the forward direction of the x axis and the oblique left direction is the forward direction of the z axis.

In the upper side of the A array oscillator 311 shown in FIG. 23, that is, the side to come into contact with the subject to be imaged, an acoustic matching layer 351 is laminated. On the acoustic matching layer 351, acoustic lenses 352 are laminated. Under the A array oscillator 311, a packing material 353 is provided. That is, the A array oscillator 311 is laminated on the packing material 353.

The acoustic lenses 352 are a lens shape such that light is concentrated along the short side 301S of the ultrasonic probe 301. According to this shape, the beam focus in the direction (the z axis direction) along the short side 301S of the ultrasonic probe 301 is realized in the A array oscillator 311. In the ultrasonic probe 301, the acoustic lenses are also formed onto the B array oscillator 312 and the C array oscillator 313 (dotted lines) which are provided on both the left and right ends of the A array oscillator 311 so as to extend this lens shape, as it is, in the positive and negative directions of the x axis.

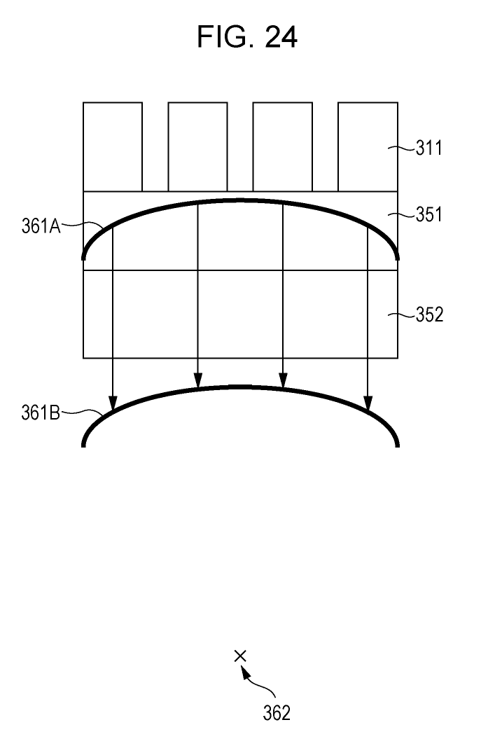

For example, in the center of the short side 301S of the ultrasonic probe 301 shown in FIG. 23, the shape of the acoustic lens 352 in the cross section cut into the vertical direction (into the xy plane) in the drawing is expressed in a flat rectangle shape as shown in FIG. 24.

According to this, in the beam forming in the x axis direction of the A array oscillator 311, a synthesis wave front 361A, which is released from the A array oscillator 311, is outputted, as a synthesis wave front 361B shown in FIG. 24, from the acoustic lens 352 without changing the shape thereof. Therefore, in this case, it is possible to ignore the effect of the acoustic lens 352.

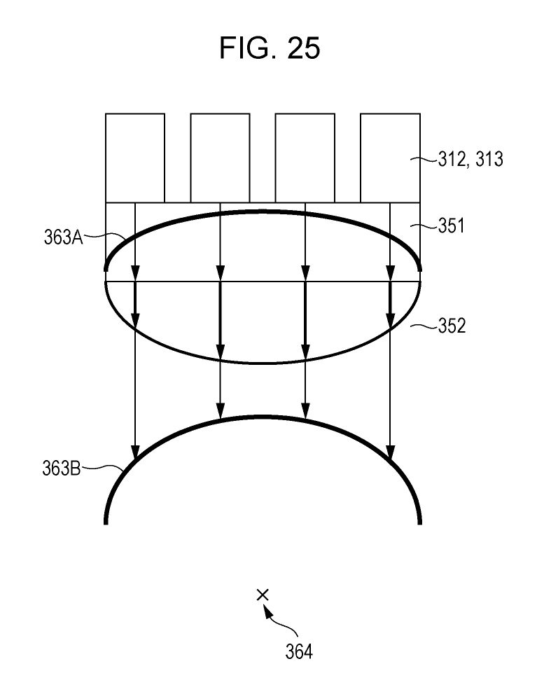

On the other hand, for example, at any position of the long side 301L of the ultrasonic probe 301 shown in FIG. 24, the shape of the acoustic lens 352 in the cross section cut into the vertical direction (into the yz plane) in the drawing is a lens shape as shown in FIG. 25. According to this, in the beam forming in the z axis direction of the B array oscillator 312 and the C array oscillator 313, the synthesis wave front 363A, which is released from the B array oscillator 312 and the C array oscillator 313, is affected to the acoustic lens 352 in the similar way to the synthesis wave front 363B shown in FIG. 25. That is to say, the synthesis wave front 363B changes R to be tight due to the lens effect of the acoustic lens 352 and thus a focal point 364 is focused at the closer position than a focal point 362 in a case of the synthesis wave front 361B of FIG. 24.

Therefore, when the beam transmission from the B array oscillator 312 and the C array oscillator 313 is performed, in consideration of the effect of the acoustic lens 352, it is necessary to perform a delay amount calculation for beam forming or the like. Even so, in the delay amount calculation for beam forming, it is only necessary to add a difference thereof and this does not lead to the enlargement of the processing amount of practical delay amount calculation or the lowering of the processing speed.

The ultrasonic probe 301 configured as described above is provided, for example, in the image processing system 201 described above with reference to FIG. 12, instead of the ultrasonic probe 111. In this case, for example, the ultrasonic wave signal form the ultrasonic probe 301 is received by the ultrasonic wave reception unit 232 and is also supplied to the sensor information acquisition unit 253, in addition to the ultrasonic image generation unit 252. A movement amount calculation process of the ultrasonic probe 301 is performed by the sensor information acquisition unit 253 as follows.

### Example of Movement Amount Calculation Process of Probe

If coordinate transformation on a general plane is considered, there is degree of freedom in parallel displacement (the x direction and the y direction), scaling, and rotation (around the y axis as a center). If the contact surface of the ultrasonic probe 301, which moves on the body surface of a human body, and the body surface of the human body are considered as a plane, it is not necessary to consider the scaling. Therefore, practically, it is preferable to know only both of the parallel displacements (the x direction and the y direction) and the rotation (around the y axis as a center).

When calculating parameters of the parallel displacement, it may be necessary to know at least one of the motions (Δx and Δz) of points. When calculating the rotational angle, it is necessary to know at least two of the motions of points. As described above, in the detection method based on two orthogonal planes which is described in Japanese Unexamined Patent Application Publication No. 2010-227603, it is merely possible to obtain the movement amount of one corresponding point.

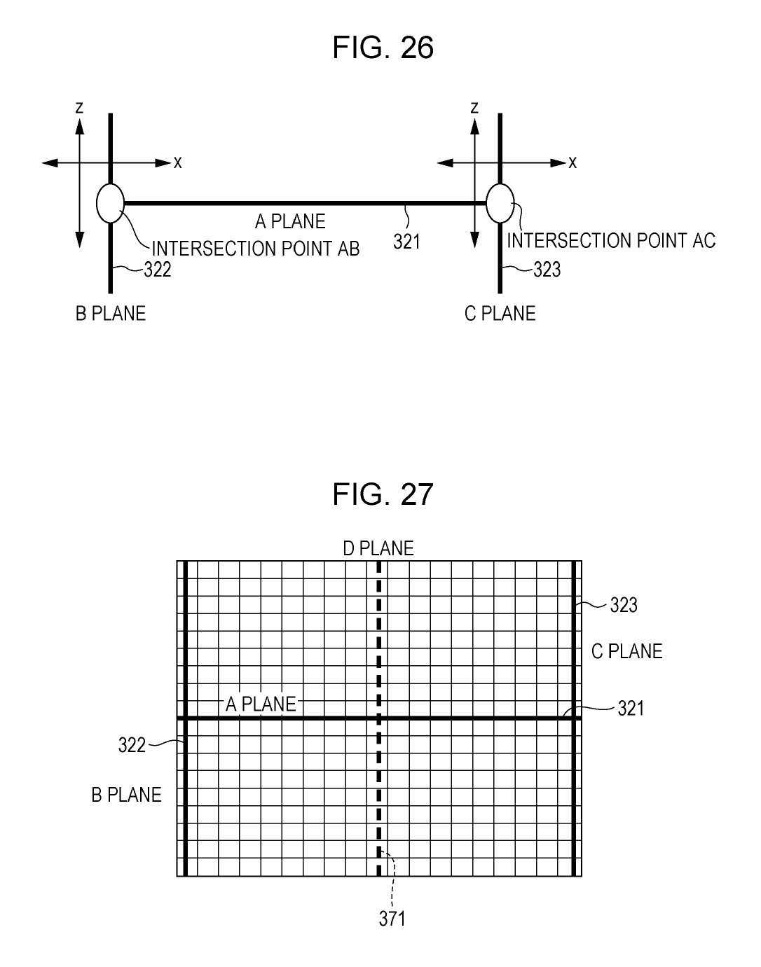

On the other hand, in the ultrasonic probe 301, as shown in FIG. 26, the A plane 321, the B plane 322 and the C plane 323 are disposed such that two intersection points (an intersection point AB and an intersection point AC) are formed on the body surface.

FIG. 26 shows a disposition example of the A plane 321, the B plane 322 and the C plane 323 of FIG. 22 when seen from the y axis direction. In the example of FIG. 26, the B plane 322 and the C plane 323 are orthogonally disposed to the A plane 321 such that the intersection point AB of the A plane 321 and the B plane 322 and the intersection point AC of the A plane 321 and the C plane 323 are formed on the zx plane.

Therefore, the sensor information acquisition unit 253, which receives the ultrasonic wave signal from the ultrasonic probe 301, can calculate the movement amount of the intersection point AB and the intersection point AC on the zx plane. According to this, it is possible to calculate the rotational angle around the center of y axis.

In the example of FIG. 26, preferably, the example in which the A plane 321, the B plane 322 and the C plane 323 are orthogonal to each other is shown. However, it is not necessary to be orthogonal to each other and the A plane 321, the B plane 322 and the C plane 323 may intersect each other (as long as it is not parallel to each other). The B plane 322 and the C plane 323 are parallel to each other but may be not parallel to each other.

The sensor information acquisition unit 253 estimates the movement amount of the ultrasonic probe 301 using an image which is reconstructed by each scanning surface (also referred to as a B mode image). The estimation method of the movement amount of the ultrasonic probe 301 is generally same as the image motion detection method. That is, between images which are respectively reconstructed at a certain time t and the next frame t+Δt, the movement amount of the intersection point AB and the intersection point AC on the image surface of whole image surface is calculated using methods such as feature point matching or block matching.

The ultrasonic images are defined by the physical feature amount of the ultrasonic probe 301 (oscillator pitch, opening size or the like), the physical feature amount of ultrasonic waves (frequency, sonic speed or the like) and signal processing after reception (frequency of AD conversion or the like). Therefore, it is possible to easily convert the movement amount (pixel number) on the image into the actual movement amount (a distant unit such as mm) in the body.

The reconstructed image becomes the xy plane in a case of the A plane 321 and becomes the yz plane in a case of the B plane 322 and the C plane 323. However, among the obtained movement amount, the movement amount in the y direction is not used in the following coordinate transformation parameter calculation. That is, respectively, (xt, zbt) and (xt+Δt, zbt+Δt) are obtained as the intersection point AB shown in FIG. 26, and (xt, zct) and (xt+Δt, zct+Δt) are obtained as the intersection point AC shown in FIG. 26.

This relationship is turned into a Helmert transformation equation so as to be developed. According to this, it is possible to obtain the movement amount (x0 and z0) of the ultrasonic probe 301 and the rotational angle θ. The Helmert transformation equation is expressed as the following equation (1).

x′=x cos θz sin θ+x0

z′=x sin θ+z cos θ+z0 (1)

The above-described movement amount calculation method can be applied to a case where a two-dimensional array probe formed by oscillators which are arranged in a two-dimensional manner as shown in FIG. 27 is used. Each grid shown in FIG. 27 represents an oscillator.

When the movement amount calculation method is applied to the two-dimensional array probe, in a similar way to the ultrasonic probe 301 according to the present disclosure having three scanning planes, a method in which the A plane 321, the B plane 322 and the C plane 323 are arranged respectively may be used or a D plane 371 represented by dotted lines may be added between the B plane 322 and the C plane 323.

It is preferable that the B plane 322, the C plane 323 and the D plane 371 be respectively orthogonal to the A plane 321 and the xz plane. However, as long as it is not parallel to the A plane 321, the above-described movement amount calculation method can be applied thereto. In the example of FIG. 27, the positional relationship between the B plane 322, the C plane 323 and the D plane 371 is merely an example and it is not necessary to be the same as the example of FIG. 27. For example, it is preferable that the B plane 322 and the C plane 323 be at both ends of the detection range, but this is not necessary.

As described above, by the signal processing method to the ultrasonic probe 301 using the ultrasonic probe 301 and the sensor information acquisition unit 253, it is possible to calculate the motion (movement parameters) of the ultrasonic probe 301. Therefore, in this case, it is not necessary to provide the movement amount sensor 121 of FIG. 12.

In the above-described explanation, the method in which images are reconstructed and thereafter the movement amount is obtained by the image matching has been described. However, a method in which the movement amount is obtained by signal processing of an RF signal at the RF signal stage before reconstructing images and then the movement amount of the ultrasonic probe 301 (in this case, a phase variation amount) is calculated based on the obtained movement amount may also be used.

Since the ultrasonic probe 301 is configured as described above, it is possible to calculate the movement amount, thereby detecting the motion of the ultrasonic probe 301 with a good accuracy. Therefore, it is possible to improve the accuracy of the application such as position presentation or panorama.

That is to say, one of the primary objects to precisely realize the position information of the probe is to make panorama (a wide viewing angle) or volume data by switching images.

In the method of the related art using the one-dimensional probe, it is possible to increase the accuracy in switching images with respect to the movement in a long axis direction (the x direction) but it is difficult to expand to a short axis direction (the z direction). A method in which a probe contact surface for creating volume data is inclined to an axis has been widely used. However, at this time, the angle is fixed (there is an instruction to perform shaking at a certain degree for a certain number of seconds) or a special system attached to an angle sensor is used.

In the method using the angle sensor, it is possible to precisely perform volume recreation up to some degree. However, since the contact surface of the probe does not move, it is not possible to create volume close to the surface skin.

According to this, by using the ultrasonic probe 301, it is possible to detect the motion of the probe with a good accuracy. Therefore, it is possible to more precisely make panorama (a wide viewing angle) or volume data by switching images.

The ultrasonic probe according to the present disclosure may be also applied to the following image processing system. Any ultrasonic probes having the above-described configurations may be used, but, as an example, the description will be made by using the ultrasonic probe 111 of FIGS. 8A to 8C.

### Fifth Embodiment

### Configuration Example of Image Processing System

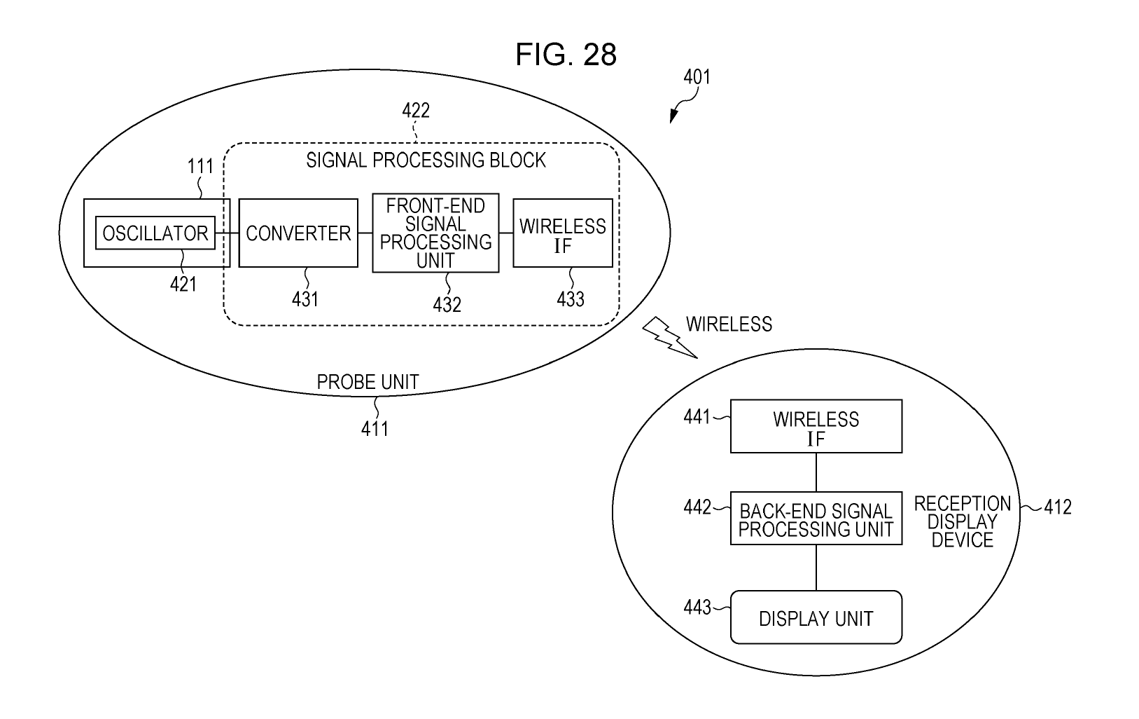

FIG. 28 is a block diagram showing a configuration example of an image processing system 401 according to the present disclosure.

The image processing system 401 shown in FIG. 28 is an apparatus that captures an image (that is, an ultrasonic image) of the inside of the subject to be imaged using ultrasonic waves to display the captured image. For example, the image processing system 401 is used for imaging of the inside of the patient's body, a fetus or the like for medical purposes, or for imaging a cross section of the inside of product or the like for industrial purposes.

The image processing system 401 is configured to include a probe unit 411 and a reception display device 412. The probe unit 411 and the reception display device 412 perform the transmission and reception of data by wireless communication, for example. The type of the wireless communication is not particularly limited as long as it ensures a sufficient bandwidth for transmitting and receiving data. The communication method is not limited to the wireless communication but may be wired communication.

The probe unit 411 is configured to include the ultrasonic probe 111 of FIGS. 8A to 8C and a signal processing block 422, for example. The ultrasonic probe 111 is a portion which is pressed onto the skin or the like of the subject to be imaged. The inside of the ultrasonic probe 111 is configured to include a plurality of oscillators 421 which are referred to as an ultrasonic transducer. The ultrasonic probe 111 is configured to include a 64ch or 128ch oscillator 421, for example. The number of the oscillators 421 included in the ultrasonic probe 111 is not limited.

The oscillator 421 transmits an ultrasonic beam to the subject to be imaged (hereinafter, also referred to as transmitted waves) based on the signal from the signal processing block 422. The oscillator 421 receives the reflective waves from the subject to be imaged (hereinafter, also referred to as received waves) and supplies the received signal to the signal processing block 422.

The signal processing block 422 is a block which processes a signal from the oscillator 421 or a signal to the oscillator 421. The signal processing block 422 is configured to include a converter 431, a front-end signal processing unit 432, and a wireless IF (InterFace) 433.

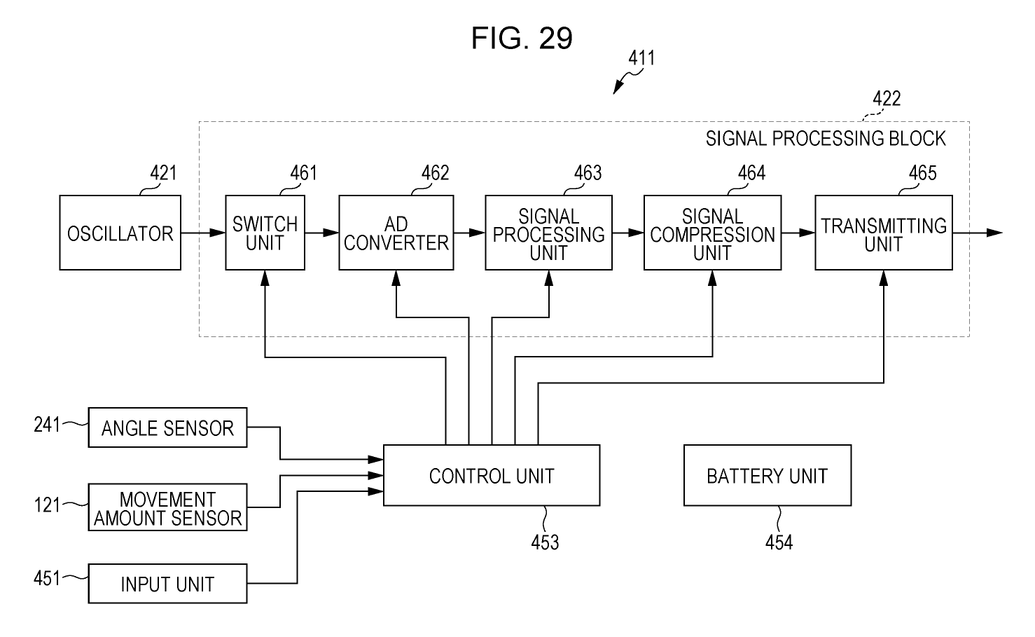

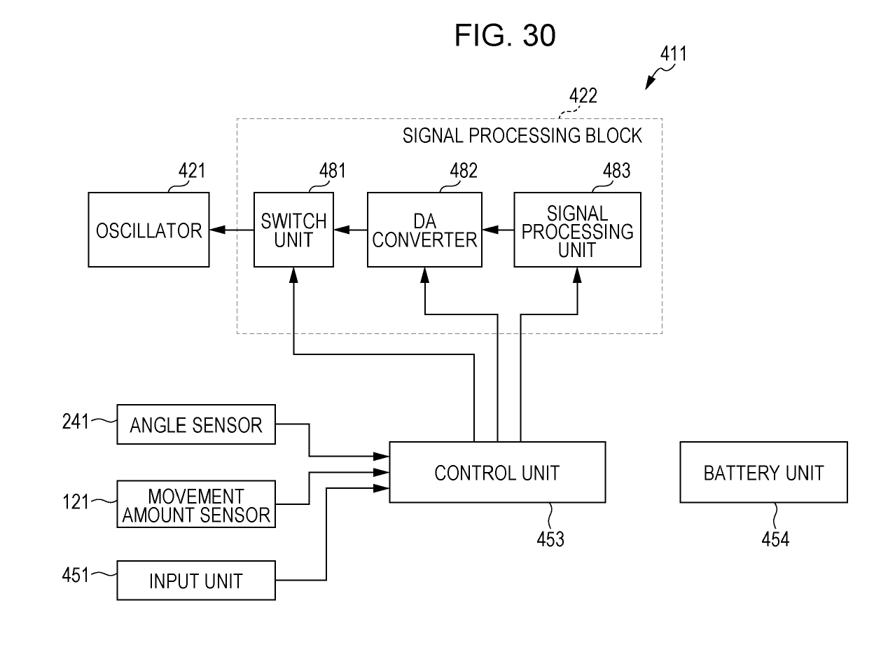

The converter 431 is configured to include an AD (Analog/Digital) converter 462 of FIG. 29 described later and a DA (Digital/Analog) converter 482 of FIG. 30 described later. The converter 431 converts the reflective waves from the oscillator 421 into digital data and supplies the converted digital data to the front-end signal processing unit 432. The converter 431 converts the digital data from the front-end signal processing unit 432 into an analog signal and supplies the converted analog signal to the oscillator 421.

The front-end signal processing unit 432 performs signal processes, such as a beam forming process, a signal compressing process, and an error correcting process, with respect to the digital data from the converter 431 and supplies the data after processing to the wireless IF 433. The front-end signal processing unit 432 generates digital data which is the source of the transmitted waves transmitted by the oscillator 421 and supplies the generated digital data to the converter 431.

The wireless IF 433 transmits the data generated from the front-end signal processing unit 432 to the reception display device 412 via wireless communication.

The reception display device 412 is configured to include a wireless IF 441, a back-end signal processing unit 442 and a display unit 443.

The wireless IF 441 receives data from the probe unit 411 and then supplies the received data to the back-end signal processing unit 442.

The back-end signal processing unit 442 decodes the compressed data transmitted from the wireless IF 441. The back-end signal processing unit 442 generates ultrasonic images showing the inside of the subject to be imaged, based on the decoded data. The back-end signal processing unit 442 supplies the generated ultrasonic images to the display unit 443.

The display unit 443 displays the ultrasonic images generated by the back-end signal processing unit 442.

In an example of FIG. 28, the configuration of the probe unit 411 is merely simplified and the description of a processing unit, a machinery part or the like, which has little relationship with the present disclosure, is omitted.

### Configuration Example of Probe Unit in Case of Receiving Side Process

FIG. 29 is a diagram showing a configuration example of the probe unit in a case where an ultrasonic wave receiving side process is performed.

In an example of FIG. 29, the probe unit 411 is configured to include the oscillator 421, the signal processing block 422, the angle sensor 241 and the movement amount sensor 121 which are included in the ultrasonic probe 111, an input unit 451, a control unit 453, and a battery unit 454.

In a case where the ultrasonic wave receiving side process is performed, the signal processing block 422 is configured to include a switch unit 461, an AD converter 462, a signal processing unit 463, a signal compression unit 464, and a transmitting unit 465. In the signal processing block 422 of FIG. 29, the signal processing unit 463, the signal compression unit 464 and the transmitting unit 465 correspond to the front-end signal processing unit 432 of FIG. 28.

The oscillator 421 receives reflective waves from the subject to be imaged and then supplies the received signal to the switch unit 461 of the signal processing block 422.

The switch unit 461 selects which signal is read among the signals received by each oscillator of the oscillator 421, under the control of the control unit 453. The oscillator 421 is configured to include, for example, 128ch oscillators. Among these, for example, when reading a 32ch signal, the switch unit 461 selects which signal out of the 32ch signals is read among 128ch oscillators. The switch unit 461 reads the selected signal and then supplies the read signal to the AD converter 462.

The AD converter 462 performs AD conversion to the signal supplied from the switch unit 461, under the control of the control unit 453. The AD converter 462 supplies the AD converted digital data to the signal processing unit 463.

The signal processing unit 463 performs the beam forming process to the digital data supplied from the AD converter 462, under the control of the control unit 453. The signal processing unit 463 also performs signal processing such as image enhancement or noise reduction to data after beam forming (hereinafter, also referred to as RF data), as necessary. The signal processing unit 463 supplies the processed data to the signal compression unit 464.

The signal compression unit 464 compresses the digital data supplied from the signal processing unit 463 in a predetermined compression format, under the control of the control unit 453. The signal compression unit 464 supplies the compressed data to the transmitting unit 465. The compression format is not limited.

The transmitting unit 465 performs adding a lengthy error correction code for transmission error compensation, or the like, to the data supplied from the signal compression unit 464 under the control of the control unit 453. Then, the transmitting unit 465 transmits the data to the reception display device 412 via the wireless IF 433 of FIG. 28. The transmitting unit 465 retransmits data to be transmitted in order to compensate a transmission error.

The angle sensor 241 and the movement amount sensor 121 are provided within the ultrasonic probe 111, as described above. The angle sensor 241 detects the rotating operation of the ultrasonic probe 111 by a user and supplies a motion parameter, which is information representing a motion feature that is the detected rotational angle of the ultrasonic probe 111, to the control unit 453. The movement amount sensor 121 detects a movement amount of the ultrasonic probe 111 by the user and supplies a motion parameter, which is information representing a motion feature that is the detected movement amount of the ultrasonic probe 111, to the control unit 453.

The input unit 451 inputs an instruction signal or the like corresponding to a user operation to the control unit 453.

The control unit 453 controls an operation of each unit configuring the signal processing block 422 depending on information detected by the angle sensor 241 and the movement amount sensor 121. As a result, it is possible to suppress power consumption to be accumulated in a battery unit 454 or to change the obtained image quality.

For example, the control unit 453 controls the switch unit 461 and changes the number of the oscillators 421 to be used for reception. In order to increase SN of reception signals, information from the plurality of oscillators 421 is generally used. For example, the channel numbers of the oscillators 421 to be used for the reception are allowed to be reduced and thus it is possible to reduce an arithmetic processing amount in the signal processing unit 463 of the subsequent stage. Therefore, it is possible to reduce the power consumption.

The control unit 453 controls, for example, the AD converter 462 and changes the sampling frequency or the bit length of digital data when the received analog signal of each channel is converted into digital data.

The image processing system 401 may be used in an image diagnostic support system CAD (Computer Aided Diagnosis) for medical purposes. When the sampling frequency is subjected to high sampling, the information amount of the obtained signal is increased and thus it is possible to perform the beam forming with higher accuracy. As a result, the image quality is improved. Therefore, the fact that sampling frequency is subjected to high sampling leads to the improvement of the diagnostic capability in CAD.

However, the fact that the frequency of the AD conversion is high cause data enlargement and thus this also affects the amount of subsequent signal processing. In a case where the image processing system 401 may not be used in CAD, that is, in a case of general diagnosis or the like, higher than good image quality is not necessary. Therefore, when lowering the sampling frequency at the time of general diagnosis or the like, it is possible to reduce the power consumption of the AD converter 462 itself and to reduce the arithmetic processing amount in the signal processing. Therefore, it is possible to reduce the power consumption. In the AD converter 462, by shortening the bit length of digital data, the same effect can be obtained as a case of lowering the sampling frequency.

For example, on an abdominal part or a chest part, when the position of the ultrasonic probe 111 approaches to a point where a user wants to view details, the user tends to move the probe unit 411 little by little and slowly in a narrow range. That is to say, when the motion of the ultrasonic probe 111 is little, the speed thereof is slow, or the movement amount thereof is small, the position of the ultrasonic probe 111 has a high possibility that the position thereof approaches to the point where the user wants to view the details. According to this, in this case, it is preferable that the image quality be as high as possible.

On the other hand, when a point where the user wants to view details is searched in a wide range, the user tends to move the probe unit 411 widely and fast in the wide range. That is to say, when the motion of the ultrasonic probe 111 is wide, the speed thereof is fast, or the movement amount thereof is great, there is a high possibility that the user is searching the point where the user wants to view details. According to this, in this case, the image quality may be lower than the case described above.

The fact described above may be applied to a joint part, in addition to an abdominal part or a chest part. Therefore, among the circumference of a joint, a place where the ultrasonic probe 111 is rotated slowly is a place where the user wants to carefully observe. According to this, the angle variation from the angle sensor 241 (that is, angular velocity) is slow, the control unit 453 controls the sampling frequency to be high such that the image quality of the ultrasonic image at the place becomes high. On the other hand, among the circumference of a joint, a place where the ultrasonic probe 111 is rotated fast is a place where the user wants to quickly observe. According to this, the angle variation from the angle sensor 241 (that is, angular velocity) is fast, the control unit 453 controls the sampling frequency to be low because the image quality of the ultrasonic image at the place may not be that high.

The control unit 453 controls, for example, the signal processing unit 463 to change parameters which relates to power, such as the number of reception focus points or the sampling frequency of RF data, among parameters at the time of performing the beam forming.

By reducing the number of reception focus points or lowering the sampling frequency of RF data, it is possible to reduce processes themselves and to reduce an amount of data over the subsequent stage. As a result, it is possible to reduce the power consumption.

The signal processing ON/OFF such as image enhancement or noise reduction in the signal processing unit 463, the control of algorithm complexity, or the like has an effect on the power. The control unit 453 may control the above-described factors.

The control unit 453 controls, for example, the signal compression unit 464 to change the compression rate. By setting the compression rate of data to be high, the amount of data for transmission from the probe unit 411 to the reception display device 412 is reduced. Therefore, it is possible to suppress the transmission power.

The control unit 453 controls, for example, the transmitting unit 465 to change the intensity or presence of adding an error correction code. By lowering the intensity of the error correction or not using an error correction function itself, it is possible to reduce the power amount necessary for transmission. The fact that the acceptance of a retransmission request of data, which is generated by cooperation with the reception display device 412, with respect to the transmitting unit 465 is changed to be rejected leads to reducing the power amount.

The battery unit 454 is formed by a rechargeable battery or the like and supplies power to each unit of the probe unit 411.

### Configuration Example of Probe Unit in Case of Transmitting Side Process

FIG. 30 is a diagram showing a configuration example of the probe unit in a case where an ultrasonic wave transmitting side process is performed.

In an example of FIG. 30, the probe unit 411 is configured to include the oscillator 421, the signal processing block 422, the angle sensor 241, the movement amount sensor 121, the input unit 451, the control unit 453, and the battery unit 454, in a similar way to the probe unit 411 of FIG. 29. The correspondent units are denoted with the correspondent reference numerals and the repeated explanation thereof is appropriately omitted.

The signal processing block 422 in a case where the ultrasonic wave transmitting side process is performed is different from the signal processing block 422 of FIG. 29 and is configured to include a switch unit 481, a DA converter 482, and a signal processing unit 483. The signal processing unit 483 of the signal processing block 422 of FIG. 30 corresponds to the front-end signal processing unit 432 of FIG. 28.

The switch unit 481 selects the oscillator 421 based on the analog signal from the DA converter 482. That is, the switch unit 481 selects a combination of oscillators to be operated among the plurality of oscillators configuring the oscillator 421. The switch unit 481 oscillates the selected oscillator 421 by connecting the selected oscillator 421 and transmitting a signal. According to this, the ultrasonic beam is transmitted from the oscillator 421 to a subject to be imaged.

The DA converter 482 converts digital data supplied from the signal processing unit 483 into an analog signal to supply the converted signal to the switch unit 481.

The signal processing unit 483 generates digital data that is the source of an ultrasonic beam, which the oscillator 421 transmits to the subject to be imaged. The signal processing unit 483 supplies the generated digital data to the DA converter 482.

In an example of FIG. 30, the control unit 453 controls the operation of each unit configuring the signal processing block 422 depending on information detected by the angle sensor 241 and the movement amount sensor 121. As a result, it is possible to suppress the power consumption accumulated in the battery unit 454 or to change the obtained image quality.

However, unlike in the case of the receiving side process of FIG. 29, in a case of the transmitting side process of FIG. 30, the switch unit 481, the DA converter 482, and the signal processing unit 483 basically co-operate with each other.

The digital data generated by the signal processing unit 483 uniquely determines a bit length of digital data passing through the DA converter 482, a sampling frequency, and the number of lines (the number of oscillators to be operated) and determines a combination of the oscillator 421 to be connected with (oscillated by) the switch unit 481.

In other words, the signal processing unit 483 uniquely determines the bit length of digital data passing through the DA converter 482, a sampling frequency, the number of lines, and a combination of the oscillator 421 connected with the switch unit 481, and generates digital data by using a combination of the determined parameters.

Therefore, in a case of the transmitting side process, the control unit 453 controls the signal processing unit 483 and changes the bit length of digital data passing through the DA converter 482, a sampling frequency, the number of lines, a combination of the oscillator 421 connected with the switch unit 481, and the like.

In the signal processing unit 483, by changing the bit length of digital data to be short or changing the sampling frequency to be low, it is possible to reduce the DA conversion process. By reducing the number of lines, it is possible to reduce the power for transmitting the ultrasonic waves.

On the other hand, in the signal processing unit 483, by changing the bit length of digital data to be long and changing the sampling frequency to be high, or increasing the number of lines, it is possible to enhance the obtained image quality.

The control unit 453 calculates the imaging angle at the time of imaging the periphery of joints based on the division number inputted by the input unit 451 and, when the angle detected from the angle sensor 241 becomes the calculated imaging angle, performs the transmission and reception of ultrasonic beam so as to generate ultrasonic images.

According to this, since the excessive transmission and reception of ultrasonic beam are not performed, it is possible to reduce the power for transmitting the ultrasonic waves.

As described above, even in both of the ultrasonic wave transmitting side process and the ultrasonic wave receiving side process, the control unit 453 controls each signal processing unit configuring the signal processing block 422. Therefore, it is possible to suppress the battery consumption of the battery unit 454 or to enhance the image quality of ultrasonic images.

Flow of Ultrasonic Wave Reception Processes

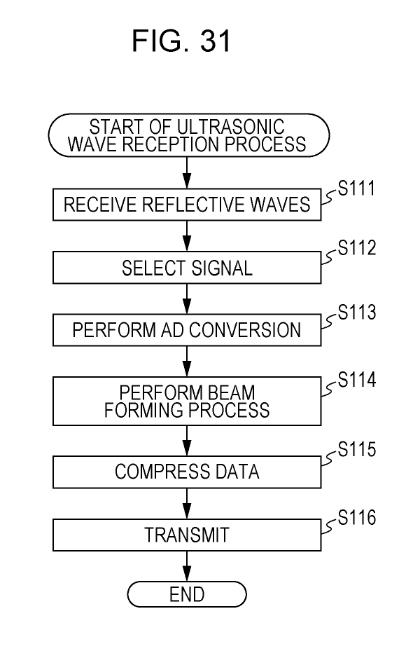

Next, with reference to a flowchart of FIG. 31, ultrasonic wave reception processes of the probe unit 411 will be described.

In Step S111, the oscillator 421 receives the reflective waves from the subject to be imaged. The oscillator 421 supplies the received signal to the switch unit 461 of the signal processing block 422.

In Step S112, the switch unit 461 selects a signal. That is, the switch unit 461 selects which signal is read among the signals received by each oscillator of the oscillator 421. At this time, the number of the reception oscillators is controlled by the control unit 453, depending on the size of the motion parameter from at least one of the angle sensor 241 and the movement amount sensor 121. The switch unit 461 reads the selected signal and supplies the read signal to the AD converter 462.

In Step S113, the AD converter 462 performs AD conversion to the signal supplied from the switch unit 461, with a predetermined sampling rate. At this time, the AD (digital data) bit length and the AD sampling rate are controlled by the control unit 453, depending on the size of the motion parameter from at least one of the angle sensor 241 and the movement amount sensor 121. The AD converter 462 supplies the AD converted digital data to the signal processing unit 463.

In Step S114, the signal processing unit 463 performs the beam forming process to the digital data supplied from the AD converter 462. The signal processing unit 463 also performs signal processing such as image enhancement or noise reduction to RF data, under the control of the control unit 453.

At this time, the frame rate and the resolution are controlled by the control unit 453, depending on the size of the motion parameter from at least one of the angle sensor 241 and the movement amount sensor 121. The processes such as image enhancement or noise reduction are also controlled by the control unit 453, depending on the size of the motion parameter from at least one of the angle sensor 241 and the movement amount sensor 121. The signal processing unit 463 supplies the processed data to the signal compression unit 464.

In Step S115, the signal compression unit 464 compresses the digital data supplied from the signal processing unit 463 in a predetermined compression format. At this time, the bit rate is controlled by the control unit 453, depending on the size of the motion parameter from at least one of the angle sensor 241 and the movement amount sensor 121. The signal compression unit 464 supplies the compressed data to the transmitting unit 465.

In Step S116, the transmitting unit 465 performs adding a lengthy error correction code for transmission error compensation, or the like, to the data supplied from the signal compression unit 464 and transmits the data to the reception display device 412 via the wireless IF 433. At this time, adding an error correction or the like is controlled by the control unit 453, depending on the size of the motion parameter from at least one of the angle sensor 241 and the movement amount sensor 121.

As described above, the data, which is subjected to signal processing with respect to the received ultrasonic waves, is transmitted from the probe unit 411 to the reception display device 412 via wireless communication.

Flow of Reception Display Processes

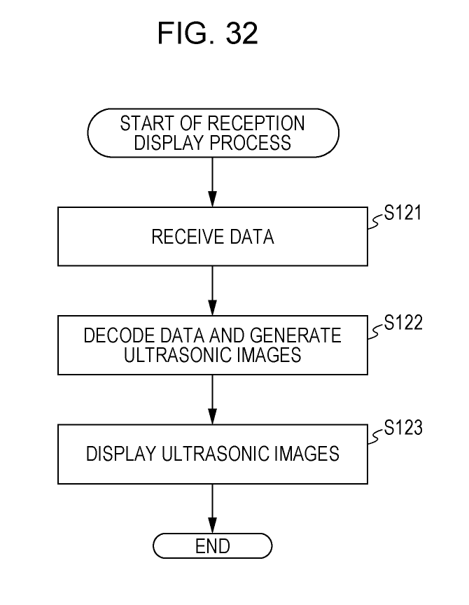

Next, with reference to a flowchart of FIG. 32, reception display processes of the reception display device 412 will be described.

In Step S121, the wireless IF 441 receives the data which is transmitted in Step S116 of FIG. 31 described above. The wireless IF 441 supplies the received data to the back-end signal processing unit 442.

In Step S122, the back-end signal processing unit 442 decodes the compressed data from the wireless IF 441 with a method corresponding to the compression of the signal compression unit 464, and generates ultrasonic images showing the inside of the subject to be imaged. The back-end signal processing unit 442 supplies the generated ultrasonic images to the display unit 443.

In Step S123, the display unit 443 displays ultrasonic images.

As described above, on the reception display device 412, ultrasonic images, which correspond to the data received by the probe unit 411 using ultrasonic waves, are displayed.

Flow of Ultrasonic Wave Transmission Processes

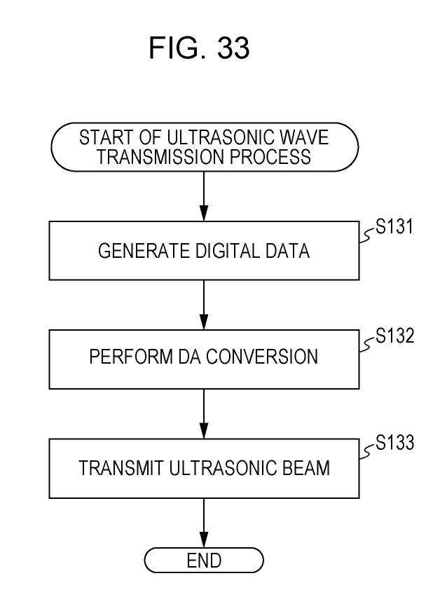

Next, with reference to a flowchart of FIG. 33, ultrasonic wave transmission processes of the probe unit 411 will be described.

In Step S131, the signal processing unit 483 generates digital data that is the source of an ultrasonic beam, which the oscillator 421 transmits to a subject to be imaged, under the control of the control unit 453.

That is, the signal processing unit 483 uniquely determines a bit length of digital data passing through the DA converter 482, a sampling frequency, the number of lines, and a combination of the oscillator 421 connected with the switch unit 481, and generates digital data by using a combination of the determined parameters. At this time, each process parameter is controlled by the control unit 453 depending on a size of motion parameter from at least one of the angle sensor 241 and the movement amount sensor 121.

The signal processing unit 483 supplies the generated digital data to the DA converter 482.

In Step S132, the DA converter 482 performs DA converting. That is, the DA converter 482 converts the digital data supplied from the signal processing unit 483 into an analog signal to supply the converted signal to the switch unit 481.

In Step S133, the oscillator 421 transmits an ultrasonic beam to a subject to be imaged. That is, the switch unit 481 selects the oscillator 421 based on the analog signal supplied from the DA converter 482. The switch unit 481 allows the selected oscillator 421 to be oscillated by connecting the selected oscillator 421 and transmitting a signal. According to this, the ultrasonic beam is transmitted from the oscillator 421 to the subject to be imaged.

In this way, in the probe unit 411, the ultrasonic beam is transmitted to the subject to be imaged.

As described above, the image quality of the ultrasonic images which is necessary for a user is recognized using the motion of the probe unit 411 (the ultrasonic probe 111) by the user. According to this, the probe unit 411 controls a process of each unit of the signal processing block 422 depending on the motion of the ultrasonic probe 111. Particularly, when the motion parameter, which represents characteristics of the motion of the ultrasonic probe 111, is large, the probe unit 411 controls to lower the image quality so as to lower the signal processing performance. On the other hand, when the motion parameter, which represents characteristics of the motion of the ultrasonic probe 111, is small, the probe unit 411 controls to enhance the image quality so as to enhance the signal processing performance.

Therefore, when a user moves the ultrasonic probe 111 slowly or little by little, for example, in order to more definitely observe a place where the ultrasonic images are imaged, using the probe unit 411, it is possible to enhance the image quality more preferentially than to suppress the power consumption.

On the other hand, when the user moves the ultrasonic probe 111 fast or widely, for example, in order to search for a point of an approximate place on the body, using the probe unit 411, it is possible to suppress the power consumption more preferentially than to enhance the image quality. In this case, even in a case where the probe unit 411 is used for diagnosis, it is possible to suppress the power consumption of the battery unit 454 in the probe unit 411. As a result, the power of the battery unit 454 can be long-lasting.

Regarding the process of the probe unit 411 described above, the example in which the angle sensor 241 is used has been described. However, instead of the angle sensor 241, an angular velocity sensor may be used.

Flow of Processes before Imaging

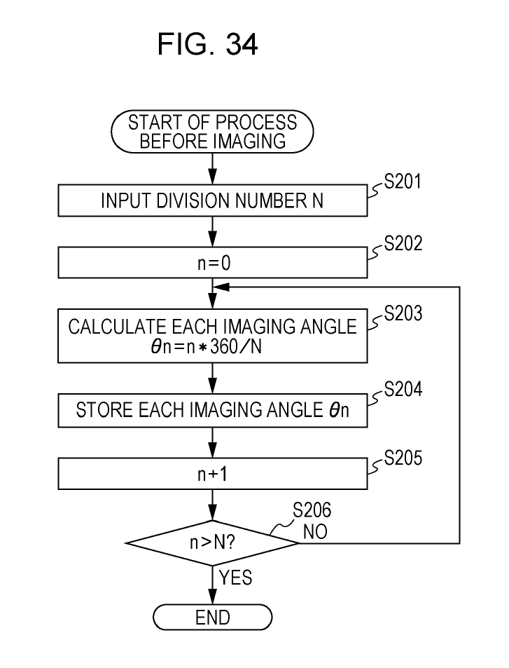

With reference to a flowchart of FIG. 34, processes before imaging in the probe unit 411 will be described.

For example, when imaging the periphery of a joint, a user inputs a division number (the number of imaging) of the periphery of a joint through the input unit 451. In accordance with this, the input unit 451 inputs a division number N to the control unit 453 in Step S201.

In Step S202, the control unit 453 sets n to be 0. In Step S203, the control unit 453 calculates each imaging angle of a joint of a diagnosis target using the division number N inputted from the input unit 451 by the following equation (2).

θn=n×360/N (2)

In Step S204, the control unit 453 stores each imaging angle θn, which is obtained in Step S203, in a built-in memory or the like. The control unit 453 sets n to be n+1 in Step S205 and determines whether or not n>N in Step S206.

When it is determined that n is not greater than N, that is, n is equal to or less than N in Step S206, the process returns to Step S203 and the subsequent processes are repeated.

When it is determined that n is greater than N in Step S206, the process before imaging is ended.

Flow of Imaging Processes

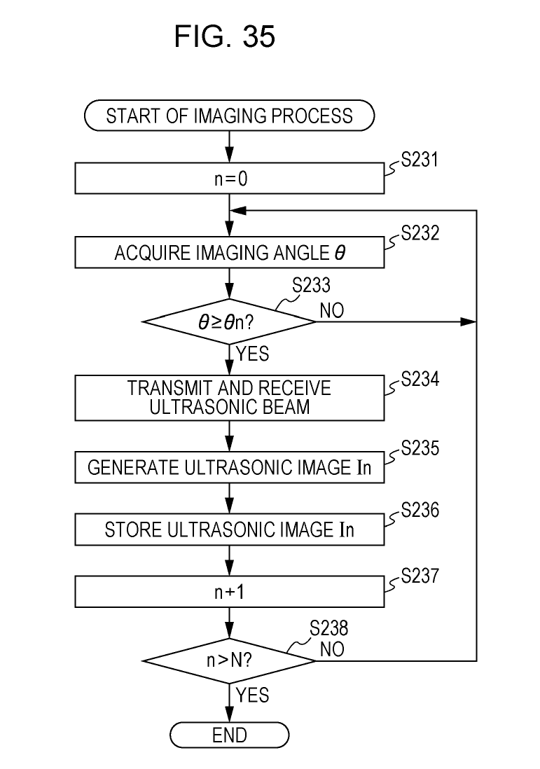

Next, with reference to a flowchart of FIG. 35, imaging processes in the probe unit 411 will be described. These processes are started, for example, when an imaging start command is inputted through the input unit 451 of the image processing system 401.

In Step S231, the control unit 453 sets n to be 0. In Step S232, the control unit 453 acquires an imaging angle θ stored in a built-in memory. The imaging angle θ is obtained by processes before imaging of FIG. 34 to be stored or is set as a default value in advance.

In Step S233, the control unit 453 determines whether or not θ≧θn. When it is determined that θ is less than θn in Step S233, the process returns to Step S232 and the subsequent processes are repeated.

When it is determined that θ is equal to or greater than θn in Step S233, the process proceeds to Step S234.

In Step S234, the control unit 453 transmits and receives an ultrasonic beam. That is to say, the control unit 453 controls the signal processing unit 483 so as to transmit and receive the ultrasonic beam when angle information detected by the angle sensor 241 becomes the imaging angle θ acquired in Step S232. In accordance with this, the ultrasonic wave transmission process which is described above with reference to FIG. 33 is performed, the ultrasonic wave reception process which is described above with reference to FIG. 32 is performed and, furthermore, Step S121 of the reception display process which is described above with reference to FIG. 32 is performed.

The back-end signal processing unit 442 generates an ultrasonic image In in Step S235 and stores the generated ultrasonic image In in Step S236.

The control unit 453 sets n to be n+1 in Step S237 and determines whether or not n>N in Step S238.

When it is determined that n is not greater than N, that is, n is equal to or less than N in Step S238, the process returns to Step S232 and the subsequent processes are repeated.

When it is determined that n is greater than N in Step S238, the process before imaging is ended.

As described above, the image processing system 401 controls the transmission timing of ultrasonic beam in accordance with the angle information. Therefore, the transmission and reception of ultrasonic beam is not performed excessively and then it is possible to reduce the power of transmitting the ultrasonic waves.

As described above, the ultrasonic probe of the present disclosure is implemented by only attaching a jig to the existing probe.

In the present disclosure, a handle having a rotational axis which has a built-in angle sensor is attached to a probe in an orthogonal manner. Therefore, an accurate angle of the probe which rotates around a cylindrical subject to be imaged can be detected.

In the present disclosure, since a guide is attached to the probe, it is possible to typically perform the transmission and reception of an ultrasonic beam from a vertical direction with respect to the subject to be imaged and to easily rotate the probe around the subject to be imaged.

In the present disclosure, since a ball joint is provided separately from the rotational axis, it is possible to improve a maneuvering feeling.

In other words, when ultrasonic images are imaged from the periphery of joints of the human body which are approximately cylindrical, by using the present disclosure, it is possible to acquire, for example, data of imaging angle and ultrasonic images for easily and precisely creating a three-dimensional structure (volume data). According to this, it is possible to achieve an imaging angle and a precise three-dimensional structure (volume data). As a result, it is possible to perform quantitative evaluation even in the observation of differences between before and after operations, the follow-up observation, or the like.

The present disclosure can be applicable to both medical purposes and non-medical purposes. When the present disclosure is applied to non-medical purposes, for example, the frequency and the intensity of the ultrasonic waves are preferably adjusted properly not to show internal organs.

The present disclosure is applicable to not only human beings but also animals, plants, artificial objects, or the like to image various cross sections of a subject to be imaged by the ultrasonic waves.

The series of processes described above may be executed by hardware or may be executed by software. In a case where the series of processes is executed by software, a program configuring the software is installed on a computer. Here, examples of the computer include a computer in which dedicated hardware is built-in, a general-purpose personal computer, for example, which is able to execute various functions by installing various programs, and the like.

### Sixth Embodiment

### Configuration Example of Computer

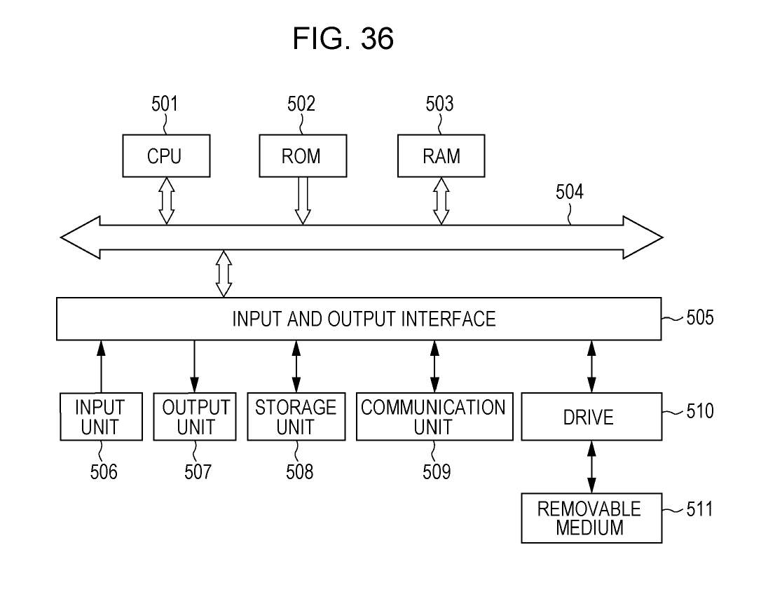

FIG. 36 is a block diagram showing a hardware configuration example of a computer which executes the aforementioned series of processing by programs.

In the computer, a Central Processing Unit (CPU) 501, a Read Only Memory (ROM) 502, and a Random Access Memory (RAM) 503 are connected to one another by a bus 504.

An input and output interface 505 is also connected to the bus 504. An input unit 506, an output unit 507, a storage unit 508, a communication unit 509, and a drive 510 are connected to the input and output interface 505.

The input unit 506 is formed of a keyboard, a mouse, a microphone, and the like. The output unit 507 is formed of a display, a speaker, or the like. The storage unit 508 is formed of a hard disk, a non-volatile memory, or the like. The communication unit 509 is formed of a network interface or the like. The drive 510 drives a removable medium 511 such as a magnetic disk, an optical disc, a magneto-optical disc, a semiconductor memory, or the like.

In the computer configured as described above, the series of processes described above is performed by the CPU 501 executing a program stored in the storage unit 508, for example, by loading the program in the RAM 503 via the input and output interface 505 and the bus 504.

The program that the computer (CPU 501) executes is able to be provided, for example, by being recorded on the removable medium 511 as a packaged medium or the like. Further, the program is able to be provided via a wired or wireless transmission medium such as a local area network, the Internet, or a digital satellite broadcast.

In the computer, the program is able to be installed on the storage unit 508 via the input and output interface 505 by fitting the removable medium 511 to the drive 510. Further, the program is able to be installed on the storage unit 508 by being received by the communication unit 509 via a wired or wireless transmission medium. Otherwise, the program may also be installed on the ROM 502 or the storage unit 508 in advance.

Here, the program that the computer executes may be a program in which processing is performed in a time series along the order described in the present specification, or may be a program in which processing is performed at necessary timings such as in parallel or when a call is made.

Here, in the present specification, a system denotes the entire apparatus formed of a plurality of devices, blocks, units or the like.

The embodiments of the present disclosure are not limited to the embodiments described above, and various modifications are possible without departing from the gist of the present disclosure.

As described above, embodiments of the present disclosure are described with reference to the attached drawings. However, the present disclosure is not limited to the embodiments. It should be understood by those skilled in the art with the knowledge in the field of the present disclosure that various modifications and alterations may occur depending on design requirements and other factors insofar as they are within the scope of the appended claims or the equivalents thereof.

Here, the present disclosure may also adopt the following configurations.

(1) An ultrasonic processing apparatus including a probe, a supporting unit that is provided at an angle perpendicular to a beam direction of the probe, a rotation mechanism that is provided between the probe and the supporting unit, and a guide that supports the rotating operation of the probe by the rotation mechanism.

(2) The ultrasonic processing apparatus according to (1), wherein the guide is provided on the probe at a right angle to a sensor surface of the probe.

(3) The ultrasonic processing apparatus according to (1) or (2), wherein the guide is provided on the probe so as to adjust a distance between the center of the sensor surface of the probe and the guide to be the radius of a diagnosis target object.

(4) The ultrasonic processing apparatus according to any one of (1) to (3), wherein the probe rotates around the rotation mechanism as an axis so as to rotate the guide in the opposite direction to the sensor surface of the probe.

(5) The ultrasonic processing apparatus according to (1), wherein the guide is provided on the same plane with a sensor surface of the probe.

(6) The ultrasonic processing apparatus according to (5), wherein the guide is provided at an angle perpendicular to the beam direction of the probe.

(7) The ultrasonic processing apparatus according to (5) or (6), wherein the guide is provided on a rotational direction side of the probe.

(8) The ultrasonic processing apparatus according to (7), wherein the guide is provided on an opposite direction side to the rotational direction of the probe.

(9) The ultrasonic processing apparatus according to (8), wherein the length of the guide in the rotational direction of the probe is longer than the length of the guide in the opposite direction side.

(10) The ultrasonic processing apparatus according to (8), wherein the length of the guide in the rotational direction of the probe is the same as the length of the guide in the opposite direction side.

(11) The ultrasonic processing apparatus according to any one of (1) to (10), wherein the supporting unit is provided on the probe so as to be at 90 degrees to the beam direction of the probe.

(12) The ultrasonic processing apparatus according to (11), wherein the supporting unit includes an auxiliary operation unit having a rotation mechanism.

(13) The ultrasonic processing apparatus according to (12), wherein the rotation mechanism of the auxiliary operation unit is prohibited from rotating about the rotational axis of the rotation mechanism.

(14) The ultrasonic processing apparatus according to any one of (11) to (13), wherein the auxiliary operation unit is detachably provided.

(15) The ultrasonic processing apparatus according to any one of (1) to (14), wherein the probe includes an angle sensor detecting an angle of the probe.

(16) The ultrasonic processing apparatus according to (15), wherein the probe includes a movement amount sensor measuring a movement amount of a sensor surface on a body surface.

(17) The ultrasonic processing apparatus according to any one of (1) to (16), further including an information acquisition unit that acquires information representing a position of the probe by which ultrasonic waves generation and reflective waves reception are performed, and a cross-sectional image generation unit that generates a tomographic image representing at least a part of the cross sections of a subject to be imaged, by arranging and synthesizing a plurality of ultrasonic images which are based on reflective waves received by the probe at a plurality of positions around the subject to be imaged, based on an angle of the probe when ultrasonic waves generation and reflective waves reception are performed.

(18) The ultrasonic processing apparatus according to (17), further including a probe state detection unit that detects a state of the probe, based on information acquired by the information acquisition unit, wherein the information acquisition unit acquires data representing the position of the probe from a plurality type of sensors, and wherein the probe state detection unit selects data to be used for detecting of the state of the probe, among data acquired by the plurality of sensors.

(19) The ultrasonic processing apparatus according to any one of (1) to (16), further including a signal processing unit that processes a signal received from a oscillator configuring the probe or a signal to be transmitted to the oscillator, and a control unit that controls a signal processing parameter so as to increase the parameter of the signal processing unit when a rotational angle of the probe is small.

(20) The ultrasonic processing apparatus according to any one of (1) to (16), further including a signal processing unit that processes a signal received from a oscillator configuring the probe or a signal to be transmitted to the oscillator, and a control unit that controls the signal processing unit so as to transmit a signal to the oscillator when a rotational angle of the probe is coincident with a predetermined imaging angle.

(21) A probe supporting apparatus including a supporting unit that is provided at an angle perpendicular to a beam direction of a probe, a rotation mechanism that is provided between the probe and the supporting unit, and a guide that supports the rotating operation of the probe by the rotation mechanism.

The present disclosure contains subject matter related to that disclosed in Japanese Priority Patent Application JP 2012-082536 filed in the Japan Patent Office on Mar. 30, 2012, the entire contents of which are hereby incorporated by reference.

It should be understood by those skilled in the art that various modifications, combinations, sub-combinations and alterations may occur depending on design requirements and other factors insofar as they are within the scope of the appended claims or the equivalents thereof.

What is claimed is:

1. An ultrasonic processing apparatus comprising:
a probe;
a supporting unit that is provided at an angle perpendicular to a beam direction of the probe;
a rotation mechanism that is provided between the probe and the supporting unit; and
a guide that supports the rotating operation of the probe by the rotation mechanism.

2. The ultrasonic processing apparatus according to claim 1, wherein the guide is provided on the probe at a right angle to a sensor surface of the probe.

3. The ultrasonic processing apparatus according to claim 2, wherein the guide is provided on the probe so as to adjust a distance between the center of the sensor surface of the probe and the guide to be the radius of a diagnosis target object.

4. The ultrasonic processing apparatus according to claim 2, wherein the probe rotates around the rotation mechanism as an axis so as to rotate the guide in the opposite direction to the sensor surface of the probe.

5. The ultrasonic processing apparatus according to claim 1, wherein the guide is provided on the same plane with a sensor surface of the probe.

6. The ultrasonic processing apparatus according to claim 5, wherein the guide is provided at an angle perpendicular to the beam direction of the probe.

7. The ultrasonic processing apparatus according to claim 6, wherein the guide is provided on a rotational direction side of the probe.

8. The ultrasonic processing apparatus according to claim 7, wherein the guide is provided on an opposite direction side to the rotational direction of the probe.

9. The ultrasonic processing apparatus according to claim 8, wherein the length of the guide in the rotational direction of the probe is longer than the length of the guide in the opposite direction side.

10. The ultrasonic processing apparatus according to claim 8, wherein the length of the guide in the rotational direction of the probe is the same as the length of the guide in the opposite direction side.

11. The ultrasonic processing apparatus according to claim 1, wherein the supporting unit is provided on the probe so as to be at 90 degrees to the beam direction of the probe.

12. The ultrasonic processing apparatus according to claim 11, wherein the supporting unit includes an auxiliary operation unit having a rotation mechanism.

13. The ultrasonic processing apparatus according to claim 12, wherein the rotation mechanism of the auxiliary operation unit is prohibited from rotating about the rotational axis of the rotation mechanism.

14. The ultrasonic processing apparatus according to claim 12, wherein the auxiliary operation unit is detachably provided.

15. The ultrasonic processing apparatus according to claim 1, wherein the probe includes an angle sensor detecting an angle of the probe.

16. The ultrasonic processing apparatus according to claim 15, wherein the probe includes a movement amount sensor measuring a movement amount of a sensor surface on a body surface.

17. The ultrasonic processing apparatus according to claim 1, further comprising:
an information acquisition unit that acquires information representing a position of the probe by which ultrasonic waves generation and reflective waves reception are performed; and
a cross-sectional image generation unit that generates a tomographic image representing at least a part of cross sections of a subject to be imaged, by arranging and synthesizing a plurality of ultrasonic images which are based on reflective waves received by the probe at a plurality of positions around the subject to be imaged, based on an angle of the probe when ultrasonic waves generation and reflective waves reception are performed.

18. The ultrasonic processing apparatus according to claim 17, further comprising:
a probe state detection unit that detects a state of the probe, based on information acquired by the information acquisition unit,
wherein the information acquisition unit acquires data representing the position of the probe from a plurality type of sensors, and
wherein the probe state detection unit selects data to be used for detecting of the state of the probe, among data acquired by the plurality of sensors.

19. The ultrasonic processing apparatus according to claim 1, further comprising:
a signal processing unit that processes a signal received from a oscillator configuring the probe or a signal to be transmitted to the oscillator; and
a control unit that controls a signal processing parameter so as to increase the parameter of the signal processing unit when a rotational angle of the probe is small.

20. The ultrasonic processing apparatus according to claim 1, further comprising:
a signal processing unit that processes a signal received from a oscillator configuring the probe or a signal to be transmitted to the oscillator; and
a control unit that controls the signal processing unit so as to transmit a signal to the oscillator when a rotational angle of the probe is coincident with a predetermined imaging angle.

21. A probe supporting apparatus comprising:
a supporting unit that is provided at an angle perpendicular to a beam direction of a probe;
a rotation mechanism that is provided between the probe and the supporting unit; and
a guide that supports the rotating operation of the probe by the rotation mechanism.
